(19) United States
(12) Patent Application Publication
(10) Pub. No.: US 2023/0304006 A1
(43) Pub. Date: Sep. 28, 2023

(54) ANTI-SEED PNAS AND MICRORNA INHIBITION

(71) Applicants: University of Connecticut, Farmington, CT (US); Beth Deacon Israel Deaconess Medical Center, Inc., Boston, MA (US)

(72) Inventors: Raman Bahal, Glastonbury, CT (US); Shipra Malik, Storrs, CT (US); Frank Slack, Waban, MA (US)

(21) Appl. No.: 18/041,271

(22) PCT Filed: Aug. 17, 2021
(86) PCT No.: PCT/US2021/046280

§ 371 Date: Feb. 10, 2023

Related U.S. Application Data
(60) Provisional application No. 63/077,038, filed on Sep. 11, 2020.
(60) Provisional application No. 63/066,539, filed on Aug. 17, 2020.

(51) Int. Cl. C12N 15/113; A61P 35/00

(52) U.S. Cl. CPC C12N 15/113; A61P 35/00; C12N 2310/3181; C12N 2310/351; C12N 2310/14

(57) ABSTRACT

Described herein is a modified anti-seed PNA including 5′-Xaa₁Xaa₂Xaa₃-N₁N₂N₃N₄N₅N₆N₇N₈ N₉-Xaa₄-3′, wherein Xaa₁, Xaa₂, Xaa₃, and Xaa₄ are each independently R or K, wherein N₁N₂N₃N₄N₅N₆N₇N₈ N₉ is a PNA which Watson-Crick base pairs to a seed sequence of an miRNA, wherein N₈ and N₉ may be null. Also included are methods of inhibiting expression of a miRNA in vivo or in vitro, and methods of treating cancer and other diseases and disorders with the modified anti-see PNAs.

### CROSS-REFERENCE TO RELATED APPLICATIONS

This application claims the priority benefit of Provisional Application Nos. 63/066,539, filed on Aug. 17, 2020, and 63/077,038, filed Sep. 11, 2020, the entire contents and disclosures of each of which are incorporated herein by reference.

### STATEMENT REGARDING FEDERALLY SPONSORED RESEARCH & DEVELOPMENT

This invention was made with government support under CA241194 awarded by the National Institutes of Health. The government has certain rights in the invention.

### INCORPORATION-BY-REFERENCE OF MATERIAL SUBMITTED ELECTRONICALLY

Incorporated by reference in its entirety herein is a computer-readable nucleotide listing submitted concurrently herewith and identified as follows: One 6,479 Byte ASCII (Text) file named “21-006_ST25.TXT,” created on Aug. 17, 2021.

### BACKGROUND

The RNA medicine field has made significant progress with the approval of: a) RNAi drugs; Onpattro® (patisiran) for treatment of polyneuropathy caused by hereditary ATTR amyloidosis and Givlaari® (givosiran) for porphyria, and b) RNA-targeting antisense drug Spinraza® (nusinersen) for treating spinal muscular atrophy. More recently, the success of milasen, an antisense drug to treat Batten's disease is another promising example of RNA-based medicine. Advances have been made in targeting messenger RNA (mRNA) for RNA-based therapies. However, therapies related to microRNA (miRNA) targeting still need to be explored. MiRNAs are short (about 22 nt to about 25 nt) non-coding RNAs that control post-transcriptional gene expression of other RNAs, especially mRNA. The 5′ seed region of miRNAs targets the 3′ untranslated region (UTR) of mRNA by homologous Watson Crick base pairing and hampers its activity by either mRNA degradation or translation inhibition. It has been well established that single miRNAs regulate the network of multiple protein-coding genes and several physiological pathways, including cell survival, differentiation, and proliferation. Therefore, an aberration of miRNA expression (upregulation or downregulation) can result in an uncontrolled cascade of cell signaling activity in the cells that can be oncogenic. miRNAs that are overexpressed in tumors (also known as oncomiRs) play an important role in promoting tumor growth, angiogenesis, and metastasis. Hence, inhibiting oncomiRs selectively using antimiR oligonucleotides have provided a new avenue for cancer therapy.

Several chemical modifications have been introduced in antimiR oligonucleotides to increase their binding affinity with cognate miRNA and to improve their enzymatic stability. 2-O-methyl oligonucleotides (e.g., antogomiRs), morpholinos, locked nucleic acid (LNA), and peptide nucleic acids (PNAs) represent important classes of synthetic nucleic acids developed to date to silence specific miRNAs via simple rules of Watson Crick (WC) base-pair recognition. Recently, some progress has been made using CRISPR/Cas9 based technology in targeting miRNAs.

Unlike other synthetic nucleic acids, PNAs are a unique class in which the phosphodiester backbone is substituted with a neutral-charge achiral N-(2-aminoethyl) glycine backbone. PNAs can bind single-strand DNA/RNA target sequences with high affinity as well as specificity and are non-susceptible to enzymatic (proteases or nucleases) degradation, making PNAs suitable for a myriad of biomedical applications like gene editing, genomic barcoding for pathogen detection, anti-infective agents and miRNA silencing. Mechanistically. PNAs bind to target miRNAs by WC base pairing and inhibit their function by sterically blocking miRNA-mRNA interaction.

Extensive translational applications of PNAs have been stymied by their limited intracellular delivery due to the neutral-charge backbone. A few prior studies noted that poly-arginine conjugated PNAs and guanidinium modified gamma PNAs undergo uptake in mammalian cells. While these strategies show promise for lab-scale setups, important issues such as; 1) cytotoxicity associated with direct exposure to cationic guanidinium domains, 2) endosomal entrapment restricting PNA's availability to the target site, and 3) challenges of large-scale production including high cost associated with optically pure gamma monomers synthesis, are yet to be resolved.

What is needed are novel methods and reagents for inhibiting miRNAs.

### BRIEF SUMMARY

In one aspect, a modified anti-seed PNA comprises

5′-Xaa₁Xaa₂Xaa₃-N₁N₂N₃N₄N₅N₆N₇N₈ N₉-Xaa₄-3′,

wherein Xaa₁, Xaa₂, Xaa₃, and Xaa₄ are each independently R or K, and wherein N₁N₂N₃N₄N₅N₆N₇N₈ N₉ is a PNA that Watson-Crick base pairs to a seed sequence of a miRNA, wherein N₈ and N₉ may be null.

In another aspect, a method of inhibiting expression of an miRNA in vivo or in vitro comprises contacting a cell with an inhibitory amount of the modified anti-seed PNA described above.

In a further aspect, a method of treating cancer comprises administering to a subject in need thereof the above-described modified anti-seed PNA.

In a yet further aspect, a method of treating cardiovascular disease, inflammation, stroke, Alzheimer's disease, schizophrenia, lysosomal storage disorders (Gaucher disease, Hurler syndrome) or progeria comprises administering to a subject in need thereof the above-described modified anti-seed PNA.

### BRIEF DESCRIPTION OF THE DRAWINGS

FIGS. 1A and B illustrates embodiments of PNAs. FIG. 1A shows the chemical structure of regular PNA, lysine conjugated PNA (KKK-PNA-K) and arginine conjugated PNA (RRR-PNA-R). FIG. 1B shows the PNA sequences of PNA1-PNA11 used to bind to miR-155 targets. PNA1-PNA3 are designed to bind seed region and PNA5 is designed to bind full length site of miR-155. PNA4 and PNA9 is a scrambled version of PNA3 and PNA8 with the same base composition, respectively. PNAs have either three lysine (K) or arginine (R) residues conjugated to N-terminus and one lysine or arginine (R) appended to C-terminus. PNAs6-11 are conjugated with 5-carboxytetramethylrhodamine (TAMRA) dye for imaging purpose. OOO represents 8-amino-2,6,10-trioxaoctanoic acid residues (mini-PEG-3). This is used to form flexible linker connecting the TAMRA and WC binding regions of the PNAs

FIG. 2 shows a dose dependent gel-shift assay of miR-155 target (1 μM) and indicated PNAs after 1 hour of incubation. Incubations were performed in simulated physiological buffer conditions (10 mM NaPi, 150 mM KCl, and 2 mM MgCl₂) for 1 hour at 37° C. followed by non-denaturing PAGE separation and SYBR™ Gold staining.

FIGS. 3A-C provide an analysis of PLGA NPs containing PNA3. FIG. 3A shows representative SEM image of PLGA NPs containing PNA3. The image was captured at 10,000× magnification. The scale bar is 4 μm. The average particle diameter (nm) and standard deviation are given for NPs. FIG. 3B shows PNA loading analysis results. FIG. 3C shows cumulative release profile data of PNA1, PNA2 and PNA3 from PLGA NPs at a given time point (shown on the X axis). N=3, data are shown as mean±standard error mean (SEM).

FIGS. 4A, B show the workflow and results for binding PNAs with target miR-155. FIG. 4A is a schematic showing the workflow to evaluate the release and in vitro binding affinity of PNAs with target miR-155. FIG. 4B shows a PAGE gel-shift assay following incubation of miR-155 target with PNA3 (perfect match), PNA4 (scramble) and PNA3, PNA4 released from PLGA NPs in simulated physiological salt conditions at 1:1 ratio. Gels were stained using SYBR™ gold.

FIG. 5 shows cellular uptake studies. Confocal images of HeLa cells after 24 hours of incubation with PLGA NPs containing PNA8. Further. DAPI was used for staining the nucleus and cell membrane was stained using MemBrite™ dye. A representative image from at least three fields is shown. Please note the concentration of only PNA8 is the same as that encapsulated in PLGA NPs (1.4 nmole of PNA equivalent dose) for comparative analysis. Blue: DAPI (nucleus), Green: cell membrane. Red: TAMRA PNA. The images were equally enhanced for clarity using ImageJ. The scale bar is 30 μm.

FIGS. 6A, B show the uptake of PNA8 into HeLa cells. FIG. 6A shows dose dependent confocal images of HeLa cells after 24 hours of incubation with PNA8 encapsulated in PLGA NPs and only PNA8. DAPI was used for nuclear staining. Please note the concentration of only PNA8 is provided based on loading of PNAs in PLGA nanoparticles. Red: PNA oligomers (TAMRA), Blue: DAPI (Nucleus). The scale bar is 10 μm. FIG. 6B is a histogram analysis of dose dependent cellular uptake of PLGA NPs containing PNA8 in HeLa cells.

FIGS. 7A, B show uptake of PNA nanoparticles in SUDHL-5 cells. FIG. 7A shows miR-155 levels in SUDHL-5 cells after treatment with PLGA NPs containing antimiR-155 PNAs at the equivalent dose of 2.5 nmol relative to normalized average control U6 (n=3, *p<0.05). AntimiR-155 (mirVana miR-155 inhibitor) transfection was used as a positive control. FIG. 7B shows dose dependent effect on SUDHL-5 cells treated with NPs containing indicated antimiR PNAs. Cell viability measured using trypan blue dye (n=3, data represented as mean±standard error mean (SEM)). For statistical analysis student t-test was used.

FIGS. 8A-E shows RNA nanoparticle delivery to cancer cells. FIG. 8A shows short TAMRA PNA (red) nanoparticles (NPs) within cryosectioned tumor cells, 4 hours, 48 hours, and 72 hours after systemic delivery. Red indicates TAMRA and Blue (DAPI) indicates nuclei, respectively. A representative image from four fields and from two different sections is shown. Scale bar represents 30 μm. FIG. 8B shows tumor growth fold change in response to systemically administered PLGA NPs containing antimiR-155 PNA3, PNA3 NPs, and PNA5 NPs. N≥5 for each group, data is shown as mean±SEM. Student t-test was used relative to control group for statistical analysis. FIG. 8C shows relative miR-155 gene expression level in RNA isolated from U2932 tumor cells from xenograft mice after systemic treatment with indicated nanoparticles. N=4 and data is shown as mean±SEM, Student t test was used for statistical analysis. *p<0.05. FIG. 8D shows gene expression level of downstream targets of miR-155; Foxo3A and Bach1 in U2932 tumor cells derived from xenograft tumor. FIG. 8E shows histopathological studies including H&E, caspase3 and TUNEL (apoptosis markers) on tumors treated with PLGA NPs containing PNA3 and control group. Representative section from each group are shown (n=6). The images were enhanced equally with ImageJ for clarity. Scale bar represents 200 μm for H&E staining, 100 μm for caspase and 30 μm for TUNEL staining. In TUNEL staining, blue (DAPI) indicates nucleus and green indicates TUNEL staining.

FIGS. 9A and B show the chemical structures and sequences of γ-PNAs. FIG. 9A shows chemical structures of regular PNA, miniPEG-γPNA and serine-γPNA. FIG. 9B shows the sequences of regular PNA1, ^{MP}γPNA 2, and ^{Ser}γPNA3 designed to bind to the seed region of miR-155. Three arginine residues (RRR) are appended on the 5′ or N-terminus and one arginine (R) is conjugated to the 3′ end or C-terminus of all the PNAs.

FIGS. 10A and B show binding studies for the γ-PNAs. FIG. 10A shows dose dependent binding of PNA1, ^{MP}-PNA2, and ^{Ser}γPNA3 after 16 hours of incubation with target miR-155 (0.1 μM) at physiological conditions. Samples were separated on a polyacrylamide gel followed by staining with SYBR gold. FIG. 10B shows time dependent binding study of PNA1, ^{MP}γPNA2, and ^{Ser}γPNA3 with target miR-155 (1 μM). PNAs (2 μM) were incubated with the target miR-155 in physiological conditions for 0.25, 1, 2, 4, 8, 12 and 24 hours followed by separation on a polyacrylamide gel. Bound and unbound fraction of DNA was stained using SYBR gold.

FIG. 11 is a schematic of the use of the modified anti-seed RNA in the treatment of cancer.

FIGS. 12A-C illustrate miR-155 gene expression and a cell viability assay. 12A is a bar graph showing miR-155 gene expression in U2932 cells relative to SUDHL-5 cells. FIG. 12B is a bar graph showing miR-155 gene expression in U2932 cells after treatment with PLGA NPs containing PNA3 (2.5 nmol). miR-155 expression relative to average control (all normalized to U6, n=3, *p<0.05, ****p<0.0005). One sample t and Wilcoxon test was used for statistical analysis. FIG. 12C is a line graph showing dose dependent cell viability assay in U2932 cells after treatment with PLGA NPs containing PNA3 and PNA4. n=3, ***p<0.0001, multiple t test was used for statistical analysis.

The above-described and other features will be appreciated and understood by those skilled in the art from the following detailed description, drawings, and appended claims.

### DETAILED DESCRIPTION

Described herein is a biocompatible nanoparticle delivery system for short PNAs (also called anti-seed reagents) with superior binding affinity that can selectively bind to the seed region of miRNA and control gene expression both in vitro as well as in vivo. Short seed targeting antimiR PNAs possess numerous advantages over full length antimiRs. During solid-phase synthesis of full-length PNAs, the growing PNA chains fold or aggregate in the neighboring chains, resulting in poor HPLC purification, low yield, and truncated side chain impurities. In contrast, short PNAs can be efficiently prepared and are easily scalable as compared to full-length antimiR PNAs. Also, short PNAs possess superior physicochemical features such as non-aggregation and high hydrophilicity as compared to full length antimiRs. Hence, due to the aforementioned merits, therapeutically active short PNAs can be comparatively easier to translate into clinical applications.

Typically, miRNAs bind mRNAs via an 8mer long stretch of nucleotide sequence called the “seed region”. The 5′ seed region of the miRNAs, are critical for stable Argonaute (AGO) binding and formation of the AGO-miRNA duplex that further activates the RNA induced silencing complex (RISC). Although the vast majority of reported antisense strategies rely on targeting full-length miRNAs, not much progress has been made in using short antimiRs. Prior studies were primarily centered on the use of tiny 8mer LNA probes to target the seed region of miRNAs and regulate gene expression. Further, short PNA probes (9-14 mer) designed to target double-stranded RNA forming a PNA:RNA2 triplex and selectively target the mRNA sequence of HOTAIR gene for cancer therapy have been developed. Similarly, we have developed short PNA probes (3mer) to target the double-stranded CUG triplet repeat-containing hairpin RNAs in vitro. Although the aforementioned short oligonucleotide probes can bind to target RNAs, few issues related to specificity, sequence selection, delivery and in vivo validation have not yet been completely resolved. In addition, short PNA-based probes have not been tested exclusively for targeting miRNAs.

Specifically, described herein are short cationic PNA probes with superior efficacy for targeting the miR-155 seed region and inhibiting its activity. miR-155 is up-regulated in many sub-types of lymphoma (including diffuse large B-cell lymphoma) and leukemia, breast, colon and lung cancers among others, and is the functional product of the B-cell integration cluster (Bic) oncogenic RNA. These studies show that miR-155 levels are useful in both diagnosis as well as prognosis of cancer.

Diffuse large B-cell lymphoma (DLBCL) represents about 30% to about 40% of all Non-Hodgkin Lymphoma (NHL) and accounts for >80% of the cases of aggressive lymphoma in the world. miRNA signatures show that miR-155 is significantly elevated in DLBCL. Without being held to theory, it is believed that the carcinogenic potential of miR-155 in DLBCL is related to its role in MYC-associated pathways contributing to the transformation of B-cells. It has been demonstrated in vitro that high levels of miR-155 assist DLBCL cells to move from G to S1 phase and inhibit apoptosis. Furthermore miR-155 also regulates the phosphatidylinositol-3 kinase (PI3K)-protein kinase B (AKT) signaling pathway inducing proliferation of DLBCL cells. miR-155 negatively regulates the expression of transcription factors FOXO3A and BACH1, both of which exhibit tumor suppressing activities. FOXO3A belongs to the family of forkhead box O (FOXO) transcription factors which regulate the expression of pro-apoptotic factors by binding to the FOXO recognition elements (FRE) in DNA, hence activating their transcription. The direct transcriptional activation of Bcl-2 family members, including Bim and bNIP3, increases mitochondrial permeability and induces apoptosis. In addition, FRE is also present in the promoter region of pro-apoptotic gene FasL which induces mitochondrial independent apoptosis. The BTB and CNC homology (BACH1) is another transcription factor which negatively regulates the expression of heme-oxygenase-1 (HO-1). HO-1 is implicated in oncogenesis and chemoresistance, and high levels of BACH1 can reduce the transcription of HO-1 gene mitigating its anti-apoptotic activity. Therefore, inhibiting oncogenic miR-155 results in upregulation of transcription factors, FOXO3A and BACH1, which induce apoptosis of DLBCL cells presenting a viable therapeutic approach for treatment of lymphomas.

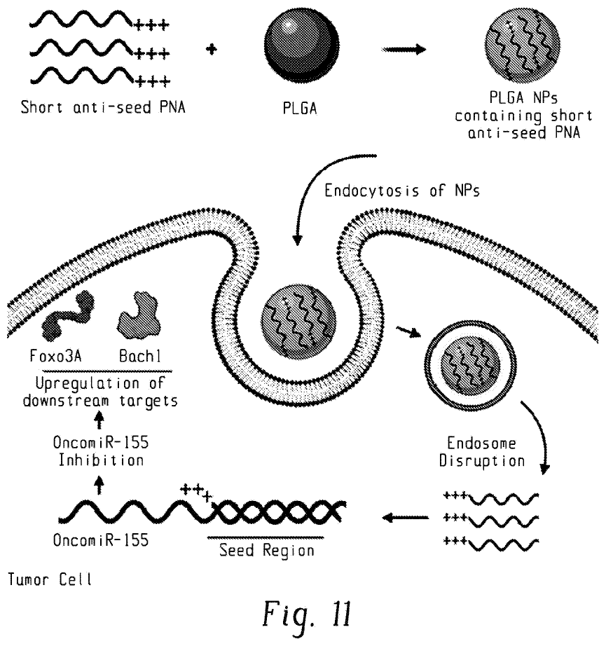

The short cationic PNA probes containing three arginine amino acids described herein were designed to bind the miR-155 seed sequences with high binding affinity to regulate its gene expression. A series of short cationic PNA probes were designed and comprehensive biophysical characterization was performed to examine their binding affinity as compared to the conventional PNAs. Further, poly(lactic-co-glycolic acid) (PLGA) based nanoparticle (NP) formulations were developed for optimal intracellular delivery of the short cationic PNA probes. PLGA has been shown to provide excellent transfection efficiency with minimal toxicity specifically for PNA delivery and is among the most effective nano-carriers reported in literature. Short cationic antimiR-155 PNAs were encapsulated in PLGA NPs a series of physico-biochemical characterizations were performed to determine optimum morphology, size distribution, surface charge, payload, and nucleic acid release profiles of NPs. Further, in vitro transfection efficiency and therapeutic efficacy of NPs were evaluated in a battery of cell culture studies. Cellular uptake of NPs was assessed by confocal and flow cytometry analyses and therapeutic efficacy was measured by performing RT-PCR and cell viability analysis in the lymphoma cell lines. To validate the results, gene expression as well as protein levels of the transcription regulators BACH1 and FOXO3A, the direct targets of miR-155, were quantified. Additionally, the safety profile of formulated PLGA NPs containing short cationic antimiR-155 PNAs was determined in primary human embryonic kidney 293 (HEK293) cells in vitro as well as in vivo in immunocompetent mice. In a xenograft mouse model, PLGA NPs containing short PNAs were able to successfully target the tumor on systemic administration. The PLGA NPs treated group showed reduced tumor growth and miR-155 levels in comparison to the control group. Furthermore, the levels of direct downstream targets of miR-155, FOXO3A and BACH1, were upregulated in the PLGA NPs treated group resulting in tumor apoptosis. Overall, the results described in this study constitute an exciting and cost-effective strategy of miRNA inhibition at the interface of synthetic nucleic acid chemistry and nanotechnology for potential cancer therapy. FIG. 11 is a schematic of the use of the modified anti-seed RNA in the treatment of cancer.

In an aspect, a modified anti-seed PNA comprises 5′-Xaa₁Xaa₂Xaa₃-N₁N₂N₃N₄N₅N₆N₇N₈ N₉-Xaa₄, wherein N₁N₂N₃N₄N₅N₆N₇N₈ N₉(SEQ ID NO: 1) is a PNA which Watson-Crick base pairs to a seed sequence of an miRNA, wherein N₈ and N₉ may be null.

In aspect, Xaa₁, Xaa₂, Xaa₃, and Xaa₄ are R, or Xaa₁, Xaa₂, Xaa₃, and Xaa₄ are K.

Exemplary miRNAs include let-7b, let-7c, let-7d, let-7e, let-7f, let-7g, let-7i, miR-100, miR-103, miR-106a, miR-107, miR-10a, miR-10b, miR-122, miR-125a, miR-125b, miR-126, miR-126*, miR-127-3p, miR-128a, miR-129, miR-133h, miR-135h, miR-137, miR-141, miR-143, miR-145, miR-146a, miR-146b, miR-148a, miR-149, miR-150, miR-155, miR-15a, miR-17-3p, miR-17-5p, miR-181a, miR-181b, miR-181c, miR-183, miR-184, miR-186, miR-187, miR-189, miR-18a, miR-190, miR-191, miR-192, miR-197, miR-199a, miR-199a*, miR-19a, miR-19h, miR-200a, miR-200a*, miR-200b, miR-200c, miR-202, miR-203, miR-205, miR-20a, miR-21, miR-210, miR-216, miR-218, miR-22, miR-221, miR-222, miR-223, miR-224, miR-23a, miR-23b, miR-24, miR-25, miR-26a, miR-26b, miR-27a, miR-27b, miR-29a, miR-29b, miR-296-5p, miR-301, miR-302a, miR-302a*, miR-30a, miR-30b, miR-30c, miR-30d, miR-30e-3p, miR-30e-5p, miR-31, miR-320, miR-323, miR-324-5p, miR-326, miR-330, miR-331, miR-335, miR-346, miR-34a, miR-370, miR-372, miR-373, miR-373*, miR-497, miR-498, miR-503, miR-92, miR-93, miR-96, and miR-99a.

Table 1 includes sequences of MiRNAs upregulated in hematological malignancies.

TABLE 1
miRNA upregulated in hematological malignancies

| | | | SEQ |
|---|---|---|---|
| | | | ID |
| Name | Cancer | Sequence | NO: |
| miR-21 | CLL, CML, | UAGCUUAUCAGACUGAUGUUGA | 5 |
| | lymphoma, | | |
| | Glioblastoma | | |
| miR-155 | CLL, B-cell | UUAAUGCUAAUCGUGAUAGGGGUU | 6 |
| | lymphoma | | |
| miR-221 | CLL | ACCUGGCAUACAAUGUAGAUUU | 7 |
| miR-888 | Endometrial, | UACUCAAAAAGCUGUCAGUCA | 8 |
| | breast, | | |
| | prostate | | |
| miR-10b | Glioblastoma, | UACCCUGUAGAACCGAAUUUGUG | 9 |
| | MDS | | |
| miR- | Lung cancer | UGGAAACAUUUCUGCACAAACU | 10 |
| 147b | | | |
| miR-122 | CTCL | UGGAGUGUGACAAUGGUGUUUG | 11 |
| miR-22 | AML | AGUUCUUCAGUGGCAAGCUUUA | 12 |
| miR-99 | AML | AACCCGUAGAUCCGAUCUUGUG | 13 |
| miR-128 | Leukemia | CGGGGCCGUAGCACUGUCUGAGA | 14 |
| miR-182 | T-ALL | UUUGGCAAUGGUAGAACUCACACU | 15 |
| miR-221 | CLL | ACCUGGCAUACAAUGUAGAUUU | 16 |
| miR-222 | CLL | CUCAGUAGCCAGUGUAGAUCCU | 17 |
| miR- | AML | CUAGGAGGCCUUGGCC | 18 |
| 4262 | | | |
| miR-20 | AML | UAAAGUGCUUAUAGUGCAGGUAG | 19 |
| miR-125 | MM, CTCL | UCCCUGAGACCCUAACUUGUGA | 20 |
| miR-17 | ALL, CML | CAAAGUGCUUACAGUGCAGGUAG | 21 |
| miR-142 | MM | CAUAAAGUAGAAAGCACUACU | 22 |
| miR- | Lymphoma | AACAUUCAACGCUGUCGGUGAGU | 23 |
| 181a | | | |
| miR-187 | Lymphoma | GGCUACAACACAGGACCCGGGC | 24 |
| miR-30 | Classical | UGUAAACAUCCUCGACUGGAAG | 25 |
| | Hodgkin | | |
| | lymphoma | | |

In an aspect, the miRNA is miR-155 and N₁N₂N₃N₄N₅N₆N₇N₈ is AGCATTAA (SEQ ID NO: 2).

In an aspect, the modified anti-seed PNA comprises detectable label, such as a 5′ TAMRA with an 8-amino-2,6,10-trioxaoctanoic acid linker.

Exemplary detectable labels include a magnetic label, a fluorescent moiety, an enzyme, a chemiluminescent probe, a metal particle, a non-metal colloidal particle, a polymeric dye particle, a pigment molecule, a pigment particle, an electrochemically active species, semiconductor nanocrystal or other nanoparticles including quantum dots or gold particles, fluorophores, quantum dots, or radioactive labels.

Exemplary fluorescent labels include rhodamine; fluorescein types including without limitation FITC, 5-carboxyfluorescein, 6-carboxy fluorescein; a rhodamine type including without limitation TAMRA; dansyl; Lissamine; cyanines; phycoerythrins; Texas Red; Cy3, Cy5, dapoxyl, NBD, Cascade Yellow, dansyl, PyMPO, pyrene, 7-diethylaminocoumarin-3-carboxylic acid and other coumarin derivatives, Marina Blue™, Pacific Blue™, Cascade Blue™, 2-anthracenesulfonyl, PyMPO, 3,4,9,10-perylene-tetracarboxylic acid, 2,7-difluorofluorescein (Oregon Green™488-X), 5-carboxyfluorescein, Texas Red™-X, Alexa Fluor® 430, 5-carboxytetramethylrhodamine (5-TAMRA), 6-carboxytetramethylrhodamine (6-TAMRA), BODIPY FL, bimane, and Alexa Fluor@ 350, 405, 488, 500, 514, 532, 546, 555, 568, 594, 610, 633, 647, 660, 680, 700, and 750, and derivatives thereof, among many others. The fluorescent label can be one or more of FAM, dRHO, 5-FAM, 6FAM, dR6G, JOE, HEX, VIC, TET, dTAMRA, TAMRA, NED, dROX, PET, BHQ, Gold540 and LIZ.

Exemplary linkers that can be used to join the detectable label and the modified PNA include 8-amino-2,6,10-trioxaoctanoic acid, an imidoester crosslinker, dimethyl suberimidate, an N-Hydroxysuccinimide-ester crosslinker, bissulfosuccinimidyl suberate (BS3), an aldehyde, acrolein, crotonaldehyde, formaldehyde, a carbodiimide crosslinker, N,N′-dicyclohexylcarbodiimide (DDC), N,N′-diisopropylcarbodiimide (DIC), 1-Ethyl-3-[3-dimethylaminopropyl]carbodiimide hydrochloride (EDC or EDAC), Succinimidyl-4-(N-maleimidomethyl)cyclohexane-1-carboxylate (SMCC), a Sulfosuccinimidyl-4-(N-maleimidomethyl)cyclohexane-1-carboxylat-e (Sulfo-SMCC), a Sulfo-N-hydroxysuccinimidyl-2-(6-[biotinamido]-2-(p-azido benzamido)-hexanoamido) ethyl-1,3′-dithioproprionate (Sulfo-SBED), 2-[N2-(4-Azido-2,3,5,6-tetrafluorobenzoyl)-N6-(6-biotin-amidocaproyl)-L-1-ysinyl]ethyl methanethiosulfonate (Mts-Atf-Biotin; available from Thermo Fisher Scientific Inc, Rockford Ill.), 2-{N2-[N6-(4-Azido-2,3,5,6-tetrafluorobenzoyl-6-amino-caproyl)-N6-(6-biot-inamidocaproyl)-L-lysinylamido]}ethyl methanethiosultonate (Mts-Atf-LC-Biotin; available from Thermo Fisher Scientific Inc), a photoreactive amino acid, an N-Hydroxysuccinimide (NHS) crosslinker, an NHS-Azide reagent (e.g., NHS-Azide, NHS-PEG4-Azide. NHS-PEG12-Azide; each available from Thermo Fisher Scientific, Inc.), or an NHS-Phosphine reagent (e.g., NHS-Phosphine, Sulfo-NHS-Phosphine; each available from Thermo Fisher Scientific, Inc.).

PNAs can be synthesized using Boc-protected monomers by methods known in the art.

In an aspect, the PNA is a gamma modified PNA.

In an aspect, the modified anti-seed PNA is encapsulated in a nanoparticle. Exemplary nanoparticles comprise liposomes, hydrogels, cyclodextrins, polylactic-co-glycolic acid (PLGA), polylactic acid (PLA), polyglycolic acid (PGA), chitosan, gelatin, polycaprolactone, a poly-alkyl-cyanoacrylate, mesoporous silica nanoparticles, poly-beta-amino-esters, polyethyleneimine (PEI) or a combination thereof.

Polymeric particles including the modified anti-seed RNAs can be made by methods including single and double emulsion solvent evaporation, spray drying, solvent extraction, solvent evaporation, phase separation, simple and complex coacervation, interfacial polymerization, and other methods well known to those of ordinary skill in the art.

A method of inhibiting expression of a miRNA in vivo or in vitro comprises contacting a cell with an inhibitory amount of the modified anti-seed PNA described herein. In an aspect, the cell is a tumor cell.

In another aspect, a method of treating cancer comprises administering to a subject in need thereof the modified anti-seed PNA described herein. Exemplary subjects include mammalian subjects such as human subjects. Exemplary cancers include lymphoma (including Diffuse Large B-Cell Lymphoma, DLBCL) and leukemia, breast, colon or lung cancer.

A method of treating cardiovascular disease, inflammation, stroke, Alzheimer's disease, schizophrenia, lysosomal storage disorders (Gaucher disease, Hurler syndrome) or progeria comprises administering to a subject in need thereof the anti-seed PNA described herein.

In certain embodiments, the modified anti-seed PNAs described herein are administered to a patient or subject. A “patient” or “subject.” used equivalently herein, means mammals and non-mammals. “Mammals” means a member of the class Mammalia including, but not limited to, humans, non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, and the like. The term “subject” does not denote a particular age or sex.

The phrase “effective amount,” as used herein, means an amount of an agent which is sufficient enough to significantly and positively modify symptoms and/or conditions to be treated (e.g., provide a positive clinical response). The effective amount of an active ingredient for use in a pharmaceutical composition will vary with the particular condition being treated, the severity of the condition, the duration of the treatment, the nature of concurrent therapy, the particular active ingredient(s) being employed, the particular pharmaceutically-acceptable excipient(s)/carrier(s) utilized, and like factors within the knowledge and expertise of the attending physician. In general, the use of the minimum dosage that is sufficient to provide effective therapy is preferred. Patients may generally be monitored for therapeutic effectiveness using assays suitable for the condition being treated or prevented, which will be familiar to those of ordinary skill in the art.

The amount of compound effective for any indicated condition will, of course, vary with the individual subject being treated and is ultimately at the discretion of the medical or veterinary practitioner. The factors to be considered include the condition being treated, the route of administration, the nature of the formulation, the subject's body weight, surface area, age and general condition, and the particular compound to be administered. In general, a suitable effective dose is in the range of about 0.1 mg/kg to about 500 mg/kg body weight per day, preferably in the range of about 5 to about 350 mg/kg per day. The total daily dose may be given as a single dose, multiple doses, e.g., two to six times per day, or by intravenous infusion for a selected duration. Dosages above or below the range cited above may be administered to the individual patient if desired and necessary.

As used herein, “pharmaceutical composition” means therapeutically effective amounts of the modified anti-seed PNA together with a pharmaceutically acceptable excipient, such as diluents, preservatives, solubilizers, emulsifiers, and adjuvants. As used herein “pharmaceutically acceptable excipients” are well known to those skilled in the art.

Tablets and capsules for oral administration may be in unit dose form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavoring or coloring agents.

For topical application to the skin, the drug may be made up into a cream, lotion or ointment. Cream or ointment formulations which may be used for the drug are conventional formulations well known in the art. Topical administration includes transdermal formulations such as patches.

For topical application to the eye, the compounds may be made up into a solution or suspension in a suitable sterile aqueous or non-aqueous vehicle. Additives, for instance buffers such as sodium metabisulphite or disodium edeate; preservatives including bactericidal and fungicidal agents such as phenyl mercuric acetate or nitrate, benzalkonium chloride or chlorhexidine, and thickening agents such as hypromellose may also be included.

The active ingredient may also be administered parenterally in a sterile medium, either subcutaneously (SC), intrathecally (IT) and intraperitoneally (IP). or intravenously, or intramuscularly, or intrasternally, or by infusion techniques, in the form of sterile injectable aqueous or oleaginous suspensions. Depending on the vehicle and concentration used, the drug can either be suspended or dissolved in the vehicle. Advantageously, adjuvants such as a local anesthetic, preservative and buffering agents can be dissolved in the vehicle.

Pharmaceutical compositions may conveniently be presented in unit dosage form and may be prepared by any of the methods well known in the art of pharmacy. The term “unit dosage” or “unit dose” means a predetermined amount of the active ingredient sufficient to be effective for treating an indicated activity or condition. Making each type of pharmaceutical composition includes the step of bringing the active compound into association with a carrier and one or more optional accessory ingredients. In general, the formulations are prepared by uniformly and intimately bringing the active compound into association with a liquid or solid carrier and then, if necessary, shaping the product into the desired unit dosage form.

The invention is further illustrated by the following non-limiting examples.

### EXAMPLES

### Materials and Methods

Materials: PNAs were synthesized using Boc-protected monomers which were purchased from ASM Chemicals and Research (Germany). TAMRA (Boc-5-carboxytetramethylrhodamine) dye was purchased from VWR (Pennsylvania). Poly(lactic-co-glycolic acid) polymer used for nanoparticle formulation was bought from Lactel Absorbable Polymers (USA). Mini-PEG-3 was obtained from Peptides International (USA). miR-155 target oligonucleotides, both 8 mer and 23 mer were purchased from Midland Certified Reagent (USA). MTS reagent and cell membrane staining dye were bought from Promega (USA) and Biotium (USA), respectively. Lysotracker™ green DND-26 was purchased from Invitrogen (#L7526). Click-iT® plus TUNEL assay (#C10617) purchased from Invitrogen was used for detecting apoptosis in tumor samples. Caspase3 antibody (#9662) was purchased from cell signaling technology. Following miR-155 targets were used during studies, miR-155 target (8mer): 5′ TTAATGCT 3′. miR-155 target (23mer): 5′ TTAATGCTAATCGTGATAGGGGT 3′ (SEQ ID NO: 26).

Synthesis of PNA Oligomers: Based on previously reported protocols, solid phase synthesis was used for synthesizing the PNAs using 4-Methylbenzhydrylamine (MBHA) resin and Boc-protected monomers. TAMRA was coupled to the N-terminus or 5′ end of the PNAs and Mini-PEG-3 was used as the linker. In order to cleave the PNAs from the MBHA resin, cleavage cocktail (m-cresol:thioanisole:trifluoromethanesulfonic acid:trifluoroacetic acid, 1:1:2:6) was used and PNA was precipitated using diethyl ether. High performance liquid chromatography (HPLC) was used for purifying the PNAs and molecular weight was confirmed using mass spectroscopy (matrix assisted laser desorption/ionization-time of flight. MALDI). The extinction coefficient of PNAs was calculated via combining the extinction coefficient of each monomer and concentration was measured using UV-Vis spectroscopy.

Gel shift assays: The binding affinity of PNAs with the target was determined as described in the art. PNAs were incubated with the target miR-155 (1 μM) at different ratios in buffer mimicking the physiological conditions at pH 7.4 and a temperature of 37° C. in the thermal cycler (Bio-Rad) over a period of one hour or 16 hours. The gels were run on polyacrylamide gel at 120V for a duration of 35 minutes. SYBR™ gold (Invitrogen) was used for visualization of bound and unbound fractions of the target.

Thermal melting analysis: Incubation of PNAs with the target miR-155, both 8 mer and 23 mer, was done in physiological conditions at a 1:1 ratio and a concentration of 4 μM. Samples were subjected to thermal cycling with the first ramp from 95° C. to 25° C., second ramp from 25° C. to 95° C. and absorbance was measured at 260 nm every 30 seconds in a UV-Vis spectrophotometer as reported in the art.

Nanoparticle formulation: Based on prior protocols, a double emulsion solvent evaporation technique was used for formulation of PLGA nanoparticles. Dichloromethane (DCM) was used to dissolve the polymer and PNAs were used at 2 nanomole/mg of polymer for encapsulation. PNA dissolved in water was added to the DCM containing polymer and ultrasonicated to form the first emulsion (w/o), which was then added to the 1 ml of polyvinyl alcohol (5% w/v, aqueous) followed by ultrasonication to form the double emulsion (w/o/w). The final emulsion was added to 10 ml of 0.3% aqueous polyvinyl alcohol and stirred overnight. Nanoparticles were washed with cold water three times to remove excess PNA or polyvinyl alcohol. The final nanoparticle pellet was resuspended in 5% (w/v, aqueous) trehalose and lyophilized overnight using the benchtop freeze dryer (Labconco). Nanoparticles obtained were weighed and covered with Parafilm®, followed by storage at −20° C.

Characterization of NPs: Nanoparticle tracking analysis (NTA) was used to measure the hydrodynamic diameter using a NanoSight NS500 (Malvern Panalytical Inc., Westborough, MA, USA) equipped with 640 nm laser. NTA measurements were taken using water as a dispersant at 25° C. For each sample three measurements were recorded for 60 seconds and the standard deviation was calculated from three measurements of each sample. The surface charge of nanoparticles was determined by measuring the zeta potential using an electrophoresis technique (Malvern Panalytical Inc, USA). The measurements were recorded at 25° C. and Smoluchowski approximation was selected in the software to obtain the final values.

Loading study: The loading of PNA in nanoparticles was determined by an organic solvent extraction technique according to protocols in the art. Nanoparticles were dispersed in dichloromethane followed by shaking at 37° C. for about 3 hours. PNAs released from the dissociated nanoparticles were extracted using equal volume of 1× Tris/EDTA buffer by shaking at 37° C. for 0.5 hour. The concentration of PNA was determined using Nanodrop™ One (ThermoFisher) in the buffer layer.

Release study: As described in a prior protocol, the release of PNA from nanoparticles was measured in phosphate buffer at pH 7.4 and 37° C. The release of PNA3 NPs was also studied at acidic pH (6.5, 5.5, and 4.5). Nanoparticles were suspended in phosphate buffer and samples were collected at different time points after centrifugation (15000 rpm, 10 mins). Nanoparticles were resuspended in fresh buffer at each time point. The concentration of PNA in the PBS samples at different time points was determined using Nanodrop™ One (ThermoFisher).

Scanning Electron Microscopy (SEM) imaging of NPs: Nanoparticles were mounted on the carbon tape and sputter coating was done for 2 minutes. Imaging of nanoparticles was done using FEI NanoSEM 450 (2.0 kV). ImageJ software was used to process the images and obtain the size distribution.

Cell culture: HeLa (ATCC® CCL-2™), SUDHL-5 (ATCC® CRL-2958™) HEK-293 (ATCC® CRL-1573™) were obtained from ATCC (USA) and UJ2932 was purchased from Leibniz Institute (DSMZ, Germany). Petri-dishes (10 cm) were used for expanding the HeLa and HEK-293 cells in presence of eagle's minimum essential medium (EMEM) which was supplemented with 10% fetal bovine serum (FBS) in the absence of antibiotics. Suspended cell lines including SUDHL-5 and U2932 were cultured in 75 cm² flasks using RPMI-1640 medium with 10% FBS without any antibiotic.

Confocal microscopy: The uptake of nanoparticles in HeLa and A549 cells was studied according to prior art protocols. The cells (50,000 to 100.000) were seeded in 12 or 24 well plates containing a glass coverslip of 1 mm thickness. Cells were incubated with nanoparticle suspension for 2, 4, 8, 12, and 24 hours. To stain the cell membrane, cells were incubated with membrane staining dye in the incubator for 5 mins. Lysosomes were stained by incubating the HeLa cells in 200 nM of Lysotracker™ at 37° C. for 40 mins. Cells were washed thoroughly with PBS and fixed in 4% PFA at room temperature. After 10 mins, cells were washed with PBS and permeabilization was done using 0.1% triton at room temperature. Cells were then washed with PBS and the coverslip was mounted on a slide using prolong diamond antifade mounting media with DAPI (Life Technologies). Images were captured using a 60× oil lens on confocal microscope (Nikon A1R).

Flow cytometry analysis: The uptake of nanoparticles in HeLa cells was quantified as reported in the artHeLa cells (100,000 cells) were seeded in 12 well plates and treated with PNA8 and PNA8 PLGA NPs. After 24 hours, cells were washed and trypsinized (Gibco, Life Technologies). Trypsinized cells were centrifuged followed by washing with PBS (2λ). The final cell pellet was then suspended in 4% PFA and analyzed using LSR Fortessa™ X-20 Cell Analyzer (BD Bioscience). The data was processed in FlowJo analysis software.

RT-PCR studies: The cells (SUDHL-5 or U2932) were treated with nanoparticles and a cell pellet was collected after 48 hours of treatment. The levels of miR-155 were quantified using the previously described protocol. The RNeasy® Mini Kit (Qiagen) was used for extraction of total RNA from the cell pellets. The cDNA was synthesized using RT primers for miR-155 (assay ID 467534_mat) and U6 (assay ID: 001973), 100 mM dNTPs, RT buffer, RNase inhibitor in T100 thermal cycler (Bio-Rad) under the conditions specified in the assay. The cDNA of miR-155 and U6 was then amplified using respective primers and universal master mix II, with UNG at conditions specified in the assay and detected using the CFX Connect Real-Time PCR detection system (Bio-Rad). For measuring the levels of FOXO3A and BACH1 mRNA, a high capacity cDNA reverse transcription kit was used, and cDNA was amplified under conditions specified in the assay (Hs00818121_m1: FOXO3A, Hs00230917_m1: BACH1). The quantification values obtained were processed via the 2° method to calculate the fold change in target gene levels.

MTT assay: HEK-293 and HeLa cells were incubated with different doses of nanoparticles in 96 well plate for 24, 48, and 72 hours. After washing with PBS (2×), cells were then incubated in fresh media containing MTS reagent (CellTiter, Promega) for an hour in the incubator (37° C., 5% CO₂). After one hour, the absorbance was read at 490 nm on iMark™ plate reader (Bio-Rad). The viability of cells was measured by calculating the fold change in absorbance of treated cells in comparison to the control.

Trypan blue assay: The cells (SUDHL-5 or U2932) were treated with nanoparticles in 96 well plate. After 48 hours, trypan blue dye was used to stain the dead cells and % cell viability was measured using the cell counter (Bio-Rad, USA).

Clonogenic assay: As previously described, HeLa cells were seeded in 24 well plate and treated with nanoparticles for 24 hours. After trypsinization, cells were counted using a cell counter (Bio-Rad, USA). Based on the cell count, equal number of treated as well as untreated cells were then passaged in 6 well plates (n=6 wells/sample). The cells were allowed to expand until the untreated cells showed colonies with more than 50 cells under the inverted microscope. The colonies were fixed using acetic acid: methanol (1:7 v/v) at room temperature and washed with PBS (2×). The colonies were then stained using 0.5% w/v crystal violet and washed under tap water after 2 hours. The colonies were allowed to dry at room temperature before counting.

Western blot analysis: SUDHL-5 cells were collected after 48 hours of treatment with 2.5 nmol PNA equivalent NPs dose. Cells were lysed using 1×RIPA buffer (Cell Signaling Technology) with protease inhibitor cocktail (Thermo Scientific) on ice for 30 min. The whole cell lysates were then clarified to remove cellular debris by centrifugation at 10,000 rpm for 10 min at 4° C. Protein concentration was measured using DC protein assay (#5000112, Bio-Rad). Equal amount of proteins (30 μg) were separated using SDS/PAGE 4-20% MP TGX Stain-Free™ gels (Bio-Rad) and transferred to PVDF membrane (Bio-Rad). After the transfer of proteins, blots were blocked using 5% milk in 1× tris-buffered saline (TBS) for one hour at RT. BACH1 protein was probed using mouse monoclonal primary antibody (sc-271211, Santa Cruz Biotechnology) at 1:200 dilution. FOXO3A protein was probed by mouse monoclonal antibody (66428-1-1g, Proteintech) at 1:100 dilution. GAPDH was used as control and was detected using mouse monoclonal GAPDH antibody (sc-47724. Santa Cruz Biotechnology) at 1:2000 dilution. The desired bands were detected using Mouse m-IgGκ BP-HRP (sc-516102. Santa Cruz Biotechnology) secondary antibody (1:3000) and immobilon western chemiluminescent HRP substrate (MilliporeSigma). Intensity of bands was measured using ImageJ 1.52a software (National Institute of Health. Bethesda, MD) and normalized against GAPDH.

Transfection of hsa-miR-155-5p inhibitor: SUDHL-5 cells were transfected with hsa-miR-155-5p inhibitor (#4464084, mirVana® miRNA inhibitors, Thermo Fisher Scientific) at a dose of 50 picomoles using reverse transfection technique. hsa-miR-155-5p inhibitor was incubated with Lipofectamine® RNAiMAX Transfection Reagent (#13778100, Invitrogen) at room temperature for 20 minutes in Opti-MEM™ reduced serum medium (#31985062. Gibco). Cell suspension was then added to the medium and incubated at 37° C. and 5% CO₂ for 48 hours. Cells were collected by centrifugation at 1000 rpm for 4 min at 4° C., followed by RNA extraction and RT-PCR to measure miR-155 levels.

In vivo studies: Female NSG mice (NOD.Cg-Prkdc^{scid}Il2rg^{tm1Wjl}/SzJ, strain 005557) were purchased from Jackson labs. Mice were maintained at Beth Israel Deaconess Medical Center (BIDMC) animal facility in accordance with the institutional animal care and use committee (IACUC) rules and guidelines. 1×10⁷ U2932 cells were injected subcutaneously on the right flank of 5-6 weeks old mice. When the tumor volume reached about 100-200 mm³ after 9-12 days, mice were divided randomly into four treatment groups (n≥5) and each group was treated with phosphate buffered saline (PBS). PNA3, PNA3 NPs, and PNA5 NPs respectively. NPs were suspended and sonicated in PBS and administered by tail vein injection. Three doses were administered over the course of 16 days. Tumor volume/size was measured with caliper every 48 hours until the tumor volume reached 2000 mm³. We did not observe any toxic effects or weight loss in the experimental group when compared to the PBS-treated group of mice. The tumor, liver and kidney harvested from these mice were stained with H&E. The tumor samples from control and PNA3 NPs treated group were also stained for Ki67, a proliferation marker and terminal deoxynucleotidyl transferase dUTP nick end labeling (TUNEL) and caspase3 for detecting apoptosis.

For biodistribution studies, NPs containing PNA-TAMRA (dye) were administered by tail vein injection when the tumor volume reached about 100-200 mm³. Mice were sacrificed by CO₂ inhalation after 4 hours, 48 hours and 72 hours of injection. Tissue samples were collected and prepared for histology. Tissue samples were fixed in OCT embedding media and sectioning was done by the Histology Core of BIDMC (Boston. MA). Tissue sections (5-10 μm of thickness) were stained with DAPI and slides were imaged using a 60× oil lens on a Nikon A1 confocal microscope.

In vivo toxicity analysis was performed with male C57BL/6J (#000664) mice of age 4-6 weeks purchased from Jackson labs. Mice were randomized into four groups (Acute toxicity: Control and PNA3 NP treated, Chronic toxicity: Control and PNA3 NP treated, n=5/group). NPs were systemically administered after resuspending in 100 μl PBS via retro-orbital injection. Control group mice were injected with 100 μl PBS. These mice were sacrificed after 8 hours of treatment for acute toxicity analysis, and 48 hours post-injection for chronic toxicity study. Plasma was collected and separated from blood by centrifugation at 5000 rpm for 10 min at 4° C. Blood chemistry analyses included creatinin, blood urea nitrogen (BUN), alkaline phosphatase, lactate dehydrogenase (LDH), alanine aminotransferase (ALT), aspartate aminotransferase (AST) and was performed by Antech diagnostics (Irvine, CA). Organs including spleen, lung, liver, heart and kidney were weighed and fixed in 10% neutral buffered formalin solution (MilliporeSigma) for more than 48 hours. Paraffin embedding of tissues followed by sectioning and staining with H&E for routine histopathology was performed.

Cytokine array: Plasma samples collected from C57BL/6J mice were analyzed to measure pro-inflammatory cytokine levels (IL-10, IL-6, IL-4, IL-2, IL-β, IFNγ using the R&D mouse pre-mixed kit (ThermoFisher Scientific). Cytokine array was performed using Luminex 200 at Clinical Research Center, UConn Health Farmington, CT.

### Example 1: Rational Design and Synthesis of AntimiR-PNA Oligomers

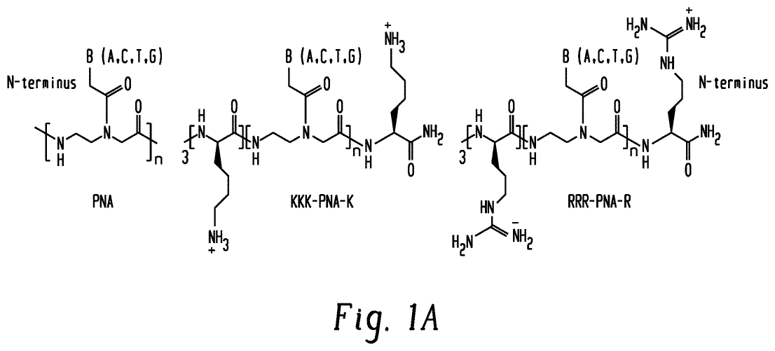

A series of PNA oligomers were synthesized as shown in FIG. 1. PNA1-3 and PNA6-8 are short length PNAs (8mer) designed to bind the seed sequence of miR-155. Seed sequences are the functional region of the miRNA that interacts with the target mRNA, subsequently activating RISC and inducing mRNA degradation. PNA1 consists of regular PNA units. PNA2 and PNA3 consist of regular PNA units conjugated with cationic residues, three lysine and arginine amino acids at N-terminus, respectively. Cationic residues were selected to increase the binding affinity of short PNAs with the target miR-155. In addition to WC base pairing between seed sequence of miR-155 and short PNA nucleobases, cationic residues on the 5′ or N termini of the short PNAs are expected to interact electrostatically with the negatively charged backbone of the non-seed region of miR-155 resulting in increased binding of miRNA-PNA hetero-duplex. Without being held to theory, only three cationic residues (arginine or lysine) were selected as excessive positive charge often leads to cytotoxicity. Full length PNA5 (with arginine) targeting miR-155 was also tested to compare its efficacy to the short length PNA3. To examine the cellular uptake properties, a series of fluorescent tetramethylrhodamine (TAMRA) dye labeled PNAs (PNA6-11) were synthesized. To decipher the role of arginine, both in NPs formulation and in cellular uptake studies, TAMRA conjugated control PNA6 and PNA10 (containing no arginine residues) were also tested. Scrambled PNAs; PNA4 and its TAMRA conjugated form (PNA9), were also synthesized. Full length TAMRA containing PNA11 (with arginine) was tested to compare its cytoplasmic delivery with short length PNA8. PNA oligomers without cationic residues result in sticky crude PNA that is difficult to purify and characterize. Hence, addition of cationic residues (lysine or arginine) at the C-terminus was performed on all the designed PNAs. All PNA oligomer sequences were synthesized, cleaved and precipitated using Boc-based synthetic protocol. PNAs were purified by RP-HPLC using water/acetonitrile based gradient solvent system (data not shown) and characterized by MALDI-TOF analysis (data not shown).

### Example 2: Binding Affinity of Short PNAs with a Synthetic MIR-155 Target

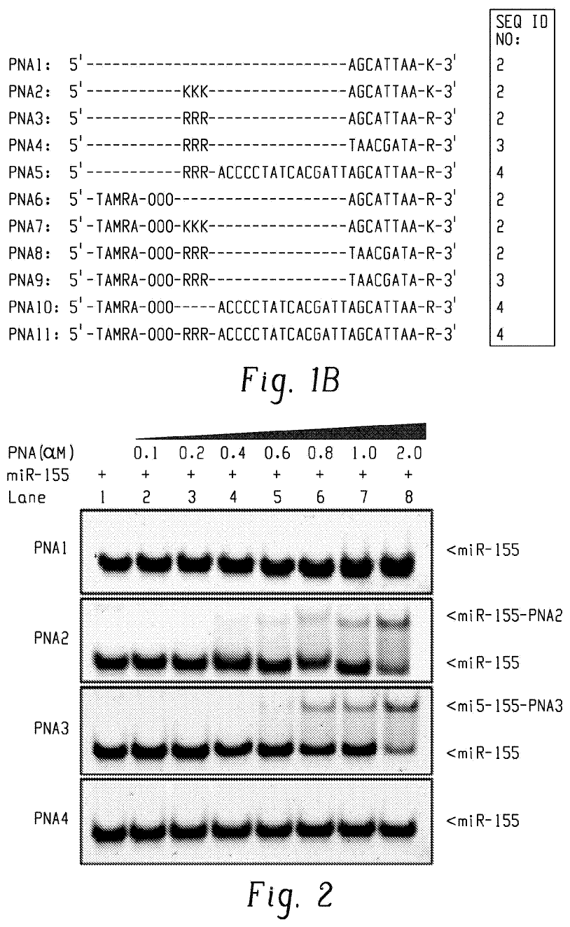

Gel shift-based assays were used to measure the binding affinity of short PNAs. Synthetic miR-155 oligonucleotides (miR-155 target) were incubated with different concentrations of PNA1, PNA2, PNA3, and PNA4 in simulated physiological buffer conditions for 1 hour and 16 hours at 37° C. (FIG. 2 and data not shown). Following this, gel shift assays were performed and the bands visualized by SYBR gold staining. As expected, we did not observe any binding with PNA1 even at PNA/miR-155 ratio of 2:1 (FIG. 2, lane 8 for PNA1). However, in the case of PNA2 and PNA3, the appearance of a higher molecular weight band (FIG. 2, lane 8 for PNA2 and PNA3) was observed.

The bound and unbound fraction for all PNAs (data not shown) was measured. PNA-miR-155 complex was most efficiently formed for PNA2 and PNA3. These results were consistent with expectations, since PNA2 and PNA3 are predicted to form a more thermodynamically stable complex due to stronger ionic interaction of cationic domains with the negatively charged backbone of miR-155 in addition to Watson-Crick base pairing. Furthermore. PNA3 was about 50% bound at 2 uM, while <40% of PNA2 was in the bound form, indicating superior binding affinity of PNA3 than PNA2 towards the target. The most plausible explanation of the finding mentioned above is that PNA3 possesses a high net positive charge due to the presence of a guanidinium group leading to its superior binding affinity as compared to lysine containing PNA2. As expected, no binding for scrambled PNA4 was observed. Similarly, in comparison to 1 hour, about 90% of miR-155 target was bound to the PNA2 and PNA3 after 16 hours of incubation (data not shown). One plausible explanation of this observation could be the difference in kinetics of binding of short antimiR probes with the miR-155 target.

### Example 3: Thermal Melting Analysis

To gain insight into the binding affinity, UV-thermal melting experiments were performed. To decipher the interaction of cationic domains of PNAs with overhanging negatively charged non-seed region of miR-155, complementary full length (23mer) and short length (8mer) miR-155 targets were used. Samples containing stoichiometric amount of PNA and miR-155 (4 uM of each) were prepared in simulated physiological buffer and annealed. Their UV absorption at 260 nm was recorded as a function of temperature. Thermal melting results with full length (23mer) miR-155 target indicated significant increase in stabilization for PNA2-miRNA-155 and PNA3-miRNA-155 duplexes with thermal denaturation temperature (Tm); about 54° C. and about 61° C. respectively as compared to regular PNA1-miR-155 complex (Tm=about 40° C.) (data not shown). However, no notice an increase in Tm for short miR-155 target (8mer) duplexes was observed. A higher Tm was observed for PNA3 in comparison to PNA2 in PNA-miR-155 complex and can be attributed to the presence of arginine domains in PNA3 that have more positive charge in comparison to lysine amino acid residues in PNA2. These results suggest that the increase in the binding affinity of PNA3 with full length target (23mer) is due to complementary WC base pairing and electrostatic interactions of the cationic domain with the negatively charged phosphate groups in the non-seed region. No melting transition was noticed for scrambled PNA4-miR-155 duplex (Tm=<30° C.).

### Example 4: Size and Surface Charge Determination of PNA Loaded PLGA NPS

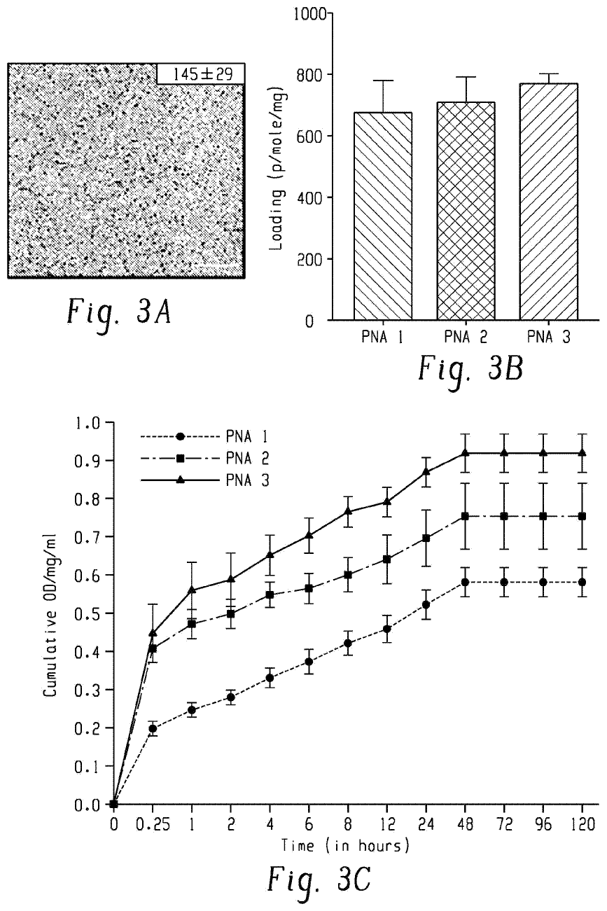

For cellular delivery, PNA oligomers were encapsulated into PLGA NPs. Prior studies have established that PLGA NPs (containing 50:50 ratio of poly-lactic acid and poly-glycolic acid) successfully deliver PNAs both ex vivo as well as in vivo. PLGA NPs were formulated using an established protocol of double emulsion solvent evaporation technique. Next, the size distribution and morphology of formulated PLGA NPs were determined using SEM. Formulated NPs demonstrated uniform size (about 145 nm) and morphology (FIG. 3A and data not shown).

The hydrodynamic diameter via nanoparticle tracking analysis (NTA) also was measured. Formulated PLGA NPs showed hydrodynamic diameter between 130-235 nm (Table 1, see above). Further surface charge on the PLGA NPs was determined by measuring the zeta potential. All PLGA NPs contained a negative zeta potential (Table 2) of −18 to −26 mV.

TABLE 2
Characterization of PLGA NPs containing PNAs
for mode diameter (Nanoparticle Tracking Analysis)
and surface charge (zeta potential).

| PLGA | Zeta | |
|---|---|---|
| polymer | potential | Mode Size (nm) ± |
| (50:50) | (mV) ± SD | SD |
| Blank NPs | −27.7 ± 2.4 | 250 ± 09 |
| PNA1 | −18.3 ± 1.0 | 207 ± 11 |
| PNA2 | −22.3 ± 1.1 | 173 ± 23 |
| PNA3 | −19.6 ± 0.5 | 211 ± 04 |
| PNA4 | −20.2 ± 1.6 | 196 ± 06 |
| PNA5 | −16.7 ± 0.5 | 229 ± 35 |
| PNA6 | −25.7 ± 1.6 | 235 ± 20 |
| PNA7 | −20.0 ± 2.9 | 129 ± 40 |
| PNA8 | −25.1 ± 1.6 | 165 ± 02 |
| PNA9 | −19.4 ± 1.2 | 209 ± 12 |
| PNA10 | −23.7 ± 4.0 | 208 ± 02 |
| PNA11 | −18.7 ± 4.9 | 213 ± 14 |

### Example 5: Loading and Release Profile Analysis of PNA from NPS

The loading of PNAs in PLGA NPs was measured using an organic solvent extraction method and absorbance was determined at 260 nm. All the NPs exhibited uniform loading between 680-810 pmole/mg of PLGA (FIG. 31). In addition, higher loading was observed for PNA3 (810 pmole/mg) as compared to PNA1 (640 pmole/mg) and PNA2 (690 pmole/mg). Without being held to theory, it is believed that this higher loading of PNA3 likely reflected the ionic interactions of its arginine residues with negatively charged PLGA polymer. Release kinetics of PNAs from NPs was determined at physiological temperature by incubating the PLGA NPs in PBS for a defined period of time and the absorbance of released nucleic acids was measured at 260 nm. All the NPs showed a slow release of PNAs (FIG. 3C and data not shown) within 48 hours. An increased PNA2 and PNA3 release possibly due to the higher loading into PLGA NPs was observed. A faster hydration of cationic PNAs in comparison to regular PNA1, which is relatively hydrophobic, was also observed.

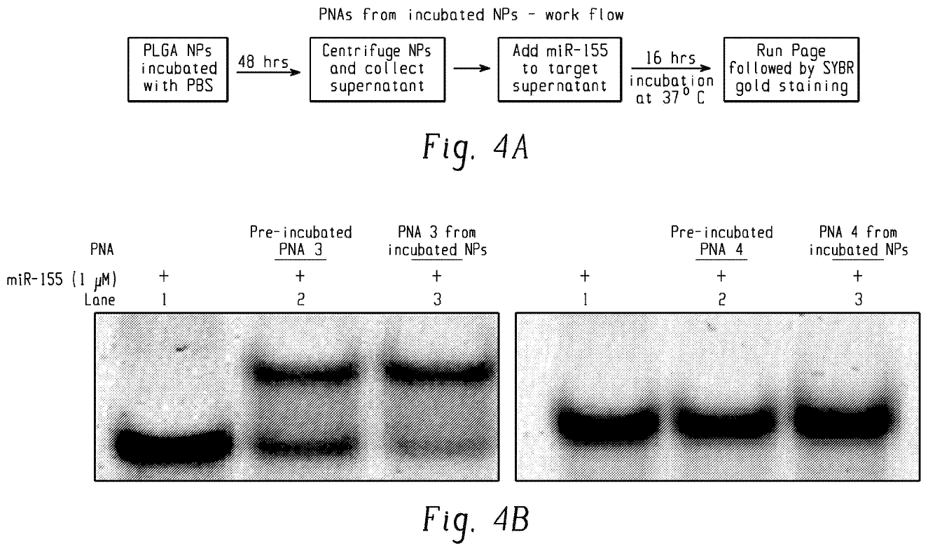

To determine the effect of pH on the release of PNA3 from NPs, we performed absorbance-based release profile studies in multiple acidic pH conditions (6.5, 5.5, and 4.5) (data not shown). A burst release at the initial time points in the acidic pH followed by a slow-release until 48 hours was observed. This could be attributed to the acid-mediated degradation of PLGA polymer. Overall, the comprehensive NP characterization established that PLGA NPs containing PNA3 are superior in loading as well as release in comparison to the rest of the formulations. Next, the integrity of PNAs1-4 after in vitro release from PLGA NPs (FIG. 4 and data not shown) was determined by gel shift binding studies with the target miR-155. The released antimiR PNA2 (data not shown) and PNA3 (FIG. 4B) were bound to miR-155 target. No binding with control PNA1 (figure data not shown) was observed whereas scrambled PNA4 (FIG. 4B) released from PLGA NPs. Together these findings suggest that PLGA NPs containing PNA3 show superior binding as well as physico-biochemical properties. Hence, cell culture based functional assays and in vivo studies were centered on PLGA NPs containing PNA3 and its TAMRA derivative (PNA8).

### Example 6: Cellular Uptake Studies

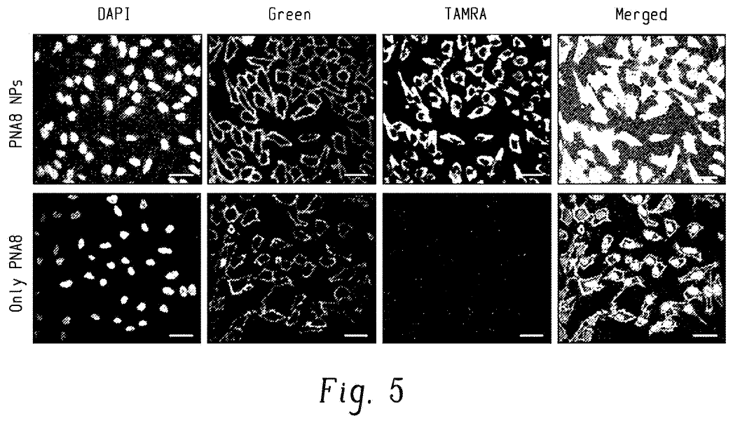

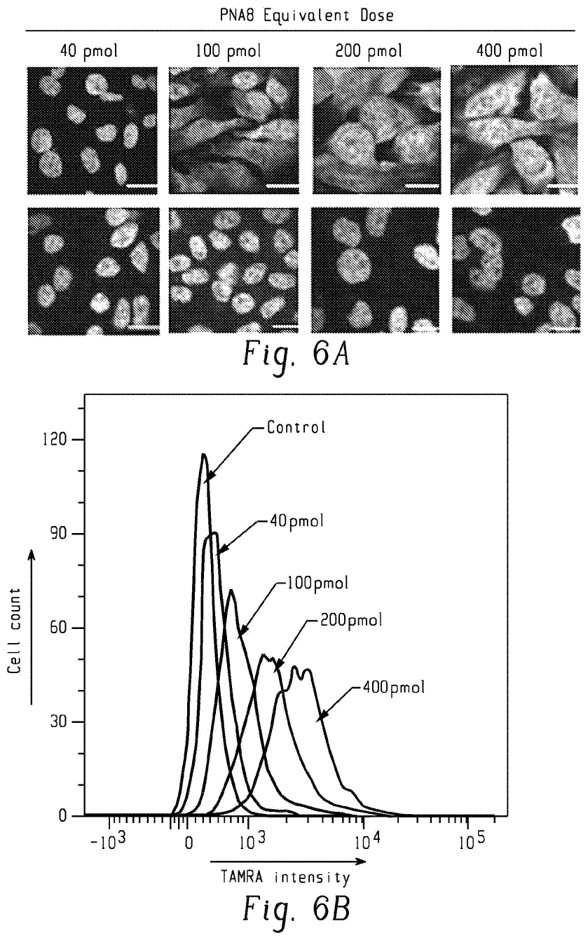

Prior to testing a miR-155 knockdown strategy, we studied the cellular uptake of PLGA NPs containing PNA8 in the cultured cells. HeLa cells cultured in logarithmic phase were incubated separately with control PNA8 and PLGA NPs containing PNA8. To compare transfection efficiency, equimolar quantities of PNA8 were used based on their loading in PLGA NPs. After incubation with HeLa cells for 24 hrs, nuclear (DAPI) and membrane staining (CellBrite™) were performed, and cells were imaged by confocal microscopy. Uniform cellular uptake of PLGA NPs containing PNA8 was noticed, as indicated by TAMRA fluorescence intensity, while no uptake was detected for PNA8 alone at the indicated concentration (FIG. 5). In order to confirm the distribution of PNA8 NPs, evaluated the uptake at multiple time points in HeLa and A549 cell lines were also evaluated (data not shown). High cellular uptake of PNA8 NPs in both the cell lines starting from 2 hours of incubation was observed. Further, confocal imaging (FIG. 6A) and flow cytometry results (FIG. 6B) confirmed the dose dependent cytosolic delivery of PLGA NPs containing PNA8 in HeLa cells.

Prior studies have shown that poly-arginine PNAs as well as guanidinium modified gamma PNAs show puncta of fluorescent signal indicating co-localization of PNAs that could be due to endosomal entrapment or non-specific interaction with other organelles. Here, it was noted that PNA8 delivered by PLGA NPs was distributed uniformly in the cytosol with relatively few puncta of TAMRA present after 24 hrs of incubation. Further, the cytosolic delivery of PLGA NPs containing other set of short PNAs (PNA6, PNA8, and PNA9) was examined (data not shown). A uniform distribution of TAMRA for PNA6 was not observed; instead, puncta of TAMRA signal were noted. In contrast, PLGA NPs containing PNA7 and PNA9 showed significant cytosolic distribution consistent with NPs containing PNA8 results. Additionally, to compare the cellular uptake of short vs full length arginine PNAs, uptake studies with PLGA NPs containing full length PNA10 (without arginine on 5′ termini) and PNA11 (with arginine residues) were performed. A similar puncta pattern of TAMRA signal for PNA10 as that of PNA6 (data not shown) was observed.

The lysosomes in HeLa cells incubated with PLGA NPs containing PNA8, PNA10, and PNA11 were further stained to determine the localization (data not shown). The majority of PNA10 accumulated in the lysosomes; however PNA8 and PNA11 (containing arginine residues) showed both lysosomal accumulation and cytoplasmic distribution. These findings together reflect that both arginine residues and the short length of PNAs in PLGA NPs significantly improve the overall intracellular cytosolic distribution in the cells.

### Example 7: Quantification of AntimiR Activity in Cell Culture

To investigate whether short PNAs can inhibit miR-155 expression in cultured cells, SUDHL-5 cells, a lymphoma cell line, were treated with PLGA NPs containing PNA1, PNA2, PNA3, and scrambled PNA4 respectively. The PLGA NPs dose was adjusted so that each group was treated with equimolar concentration of PNA. After 48 hours of treatment, miR-155 levels were quantified by RT-PCR.

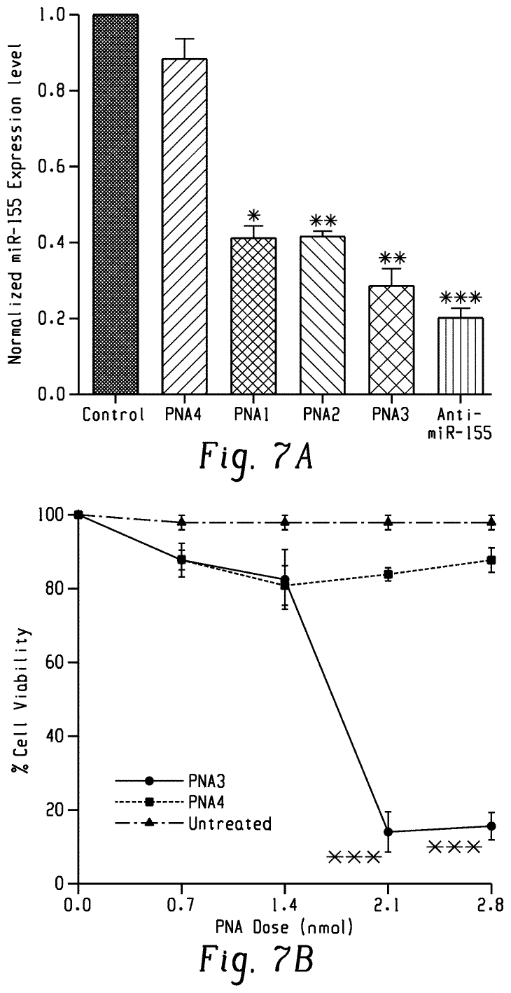

The RT-PCR results showed a about 50% and about 51% reduced miR-155 expression in the cells treated with PLGA NPs containing PNA1 and PNA2, respectively. Whereas PLGA NPs containing PNA3 treated cells showed a about 72% reduction in miR-155 expression (FIG. 7A). These results substantiate our aforementioned findings indicating that PNA3 possess superior efficacy as compared to PNA1 and PNA2 with a potency comparable to commercially available antimiR-155 (mirVana® miRNA inhibitors). In contrast, no decrease in miR-155 expression was observed in the SUDHL-5 cells treated with PLGA NPs containing scrambled PNA4.

To test whether miR-155 inhibition could also affect its downstream targets in SUDHL-5 cells, the levels of FOXO3A and BACH1 were measured and targets of miR-155 were predicted by RT-PCR analysis as well as by western blotting. miR-155 upregulation decreases FOXO3A and BACH1 levels. Using RT-PCR, about a 40% increase in BACH1 and about a 20% increase in FOXO3A gene expression was found after treatment with PLGA NPs containing PNA3 (data not shown). Western blot analysis was then perform, and consistent with the above gene expression results, about about a 30% increase in BACH1 and about a 21% increase in FOXO3A protein levels (data not shown) was observed. It was also observed that the miR-155 inhibition by PLGA NPs containing PNA3 leads to reduced SUDHL-5 viability in a dose dependent manner as compared to cells treated with PLGA NPs containing scrambled PNA4 (FIG. 7B). Next, miR-155 inhibition activity of PLGA NPs containing short length PNA3 was evaluated as compared to PLGA NPs containing full length PNA5 in treated SUDHL-5 cells.

The cells treated with an equimolar concentration of PNAs in PLGA NPs resulted in about a 75% decrease in miR-155 level with PNA5 and about a 60% decrease in cells treated with short PNA3 (data not shown). The levels of miR-155 downstream targets (FOXO3A and BACH1) in the cells treated with PNA5 and PNA3 containing NPs was also assessed (data not shown). Collectively, these results indicated that short cationic PNA3 is relatively comparable in targeting miR-155 vis-à-vis to full-length PNAs.

Similarly, in addition to PNA equivalent dose effect as mentioned above, it was also determined miR-155 expression at equivalent doses of PLGA NPs containing antimiR-155 PNAs instead of PNA equivalent dose. Consistent with prior findings, RT-PCR results showed an about an 80% decrease in miR-155 levels in the cells treated with PNA3-containing PLGA NPs (data not shown). Consistent with miR-155 gene expression results, a 2-fold increase in FOXO3A mRNA levels and 3-fold increase in BACH1 mRNA levels in SUDHL-5 cells treated with PLGA NPs containing PNA3 (data not shown) was also observed.

Further, the cell viability results were confirmed by clonogenic assay. A clonogenic assay was performed on adherent HeLa cells instead of non-adherent SUDHL-5 cells. The cells were treated with PNA3 and PNA4 NPs. After 7 days, colonies were stained with crystal violet and counted for further analysis. PNA3 NPs treated HeLa cells showed about about a 50% reduction in colonies in comparison to the control group and cells treated with PNA4 NPs (data not shown).

Further, an MTT assay was performed on embryonic kidney cells (HEK293 cells) which are commonly used for cytotoxicity analysis, after 24 hrs, 48 hrs, and 72 hrs of treatment with PLGA NPs containing PNA3 at different doses to assess the in vitro safety. As expected PNA3 NPs treated HEK293 cells showed no signs of cytotoxicity at stated dose or time point data not shown). In contrast, in HeLa cells that show overexpression of miR-155, we noted a dose-dependent decrease in cell viability (data not shown).

### Example 8: Systemic Administration of PLGA NPS Cause Tumor Growth Inhibition

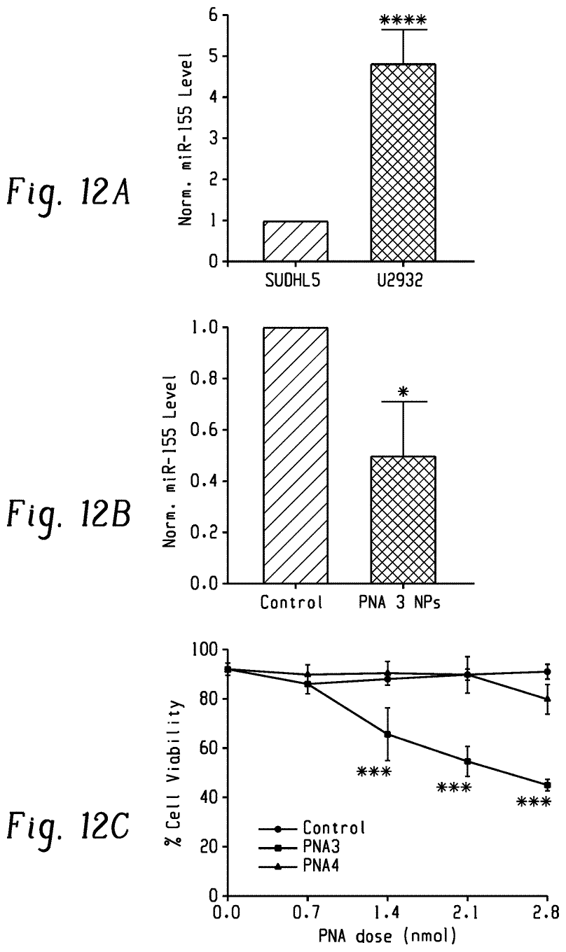

In prior work, it has been established that U2932 (another lymphoma cell line) cells reproducibly generate ectopic xenograft mouse models and easily induce tumors in mice. Henceforth, U2932 cells were chosen for establishing xenograft tumor mice model for in vivo studies. miR-155 expression levels were analyzed in U2932 cells. Greater than a 4-fold upregulation of miR-155 in U2932 was observed as compared to SUDHL-5 cells (FIG. 12A). Further, prior to in vivo studies, U2932 cells treated with PLGA NPs containing PNA3 resulted in about a 50% reduction in miR-155 levels (FIG. 12B). In addition, PLGA NPs containing PNA3 caused considerable decrease in cell viability of U2932 cell lines in dose dependent manner (FIG. 12C).

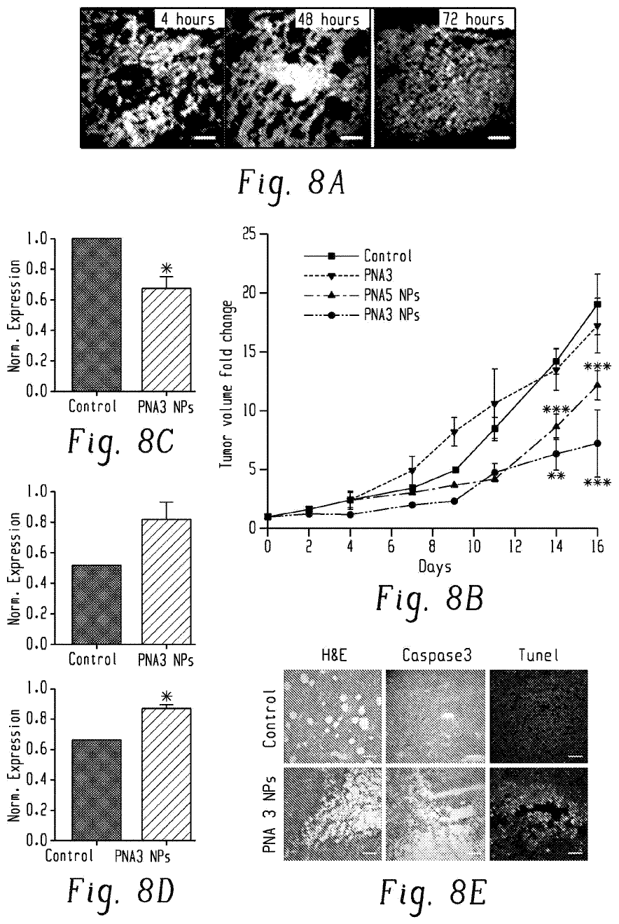

First, to assess the distribution in vivo, PLGA NPs containing TAMRA PNA were administered systemically and localization of fluorescent TAMRA signal was assessed after 4 hrs, 48 hrs, and 72 hrs in tumor (FIG. 8A), liver, kidney, and spleen (data not shown) by confocal microscopy. Significant distribution of PNA-TAMRA within the sectioned tumor cells in comparison to other organs after 4 hrs, 48 hrs, and 72 hrs of systemic treatment was confirmed. Furthermore, the maximum distribution of TAMRA was noticed in the tumor after 48 hrs of systemic treatment.

The impact on tumor growth upon systemic treatment with only PNA3, PLGA NPs containing short PNA3 and NPs containing full length PNA5 in comparison to control vehicle treated group was examined. For tumor growth delay assay, 2.4 mg/kg of PNA3 either alone or encapsulated in PLGA NPs were administered systemically. Tumor growth was assessed 3 times per week (data not shown). No significant changes in weights of the mice (data not shown) were observed. However, mice treated with PNA3 and PNA5 containing PLGA NPs showed a decrease in tumor growth as compared to only PNA3 or the control group (FIG. 8B). Further, PNA5 NPs showed similar reduction in tumor growth as that of PNA3 NPs. By day 16, control vehicle treated tumors showed an average increase of about a 15-fold in the tumor volume in comparison to PNA3 NPs and PNA5 NPs which showed only about a 6-fold and about a 10-fold increase at the same time point.

Further, the miR-155 expression level in RNA isolated from the in vivo treated U2932 tumor cells was assessed. As shown in FIG. 8C, the PLGA containing short PNA3 treated tumors showed a about a 35% decrease in the miR-155 levels as compared to control group. The gene expression level of miR-155 targets was also examined; BACH1 and FOXO3A and noticed that short PNA3 treated U2932 tumors showed increased expression of BACH1 and FOXO3A (FIG. 8D). Tumor histopathology to correlate microscopic findings with the macroscopic response was also examined. In addition to the smaller overall size of the tumors treated with PNA3, the histopathology (H&E) of treated tumors showed extensive replacement of tumor with adipose tissue and connective tissue in the PLGA NPs containing PNA3 treated tumors (FIG. 8E), whereas control vehicle treated tumors showed confluent tumor without evidence of significant intra-tumoral adipose or connective tissue. The histologic changes observed in the treated tumors may be due to decrease in miR-155 levels in tumor cells. In order to determine proliferation in tumors treated with PNA3 NPs, Ki67 staining was performed. Reduced staining of Ki67 antigen in PNA3 NPs treated tumors was observed, which may indicate low proliferation in comparison to the control group (data not shown). Further, TUNEL assay and caspase3 staining (FIG. 8E) confirmed significantly higher apoptosis in PNA3 NPs treated tumors in comparison to the control group.

### Example 9: Systemic Administration of PLGA NPS Containing Short PNAs is not Toxic

Next, acute and chronic toxicity profile was tested for in immunocompetent mice after systemic delivery of PLGA NPs containing PNA3. Groups of mice were euthanized at two different time intervals; 8 hrs (acute) and 48 hrs (chronic) after systemic delivery to determine acute and chronic toxicity, respectively (data not shown). Overall, after administration of PLGA NPs containing short PNA3, no gross acute and chronic toxicity was noted in treated mice, including skin reactions, or behavioral changes. Furthermore, an evaluation of blood chemistries did not reveal any signs of renal or hepatic damage (data not shown). No significant weight changes of the mice (data not shown) and other major organs for PLGA NPs treated and control group for acute as well as chronic toxicity studies were observed (data not shown). Histological analysis was also performed in liver and kidney to corroborate these findings. No signs of toxicity in histology of liver and kidney as compared to control group were observed (data not shown). The immunogenicity of PLGA NPs containing PNA3 was evaluated by cytokine array analysis. Six different serum cytokine levels in the mice treated with PLGA NPs in comparison to control groups for acute as well as chronic toxicity studies data not shown). These results are consistent with the lack of an immune response. Together these results do not indicate any normal tissue toxicity with PLGA NPs containing short PNA3 treatment.

Further, histological analysis was performed on liver and kidney of NSG mice (data not shown) used in efficacy studies where multiple doses of PNA3 NPs were administered to check for any toxic response. No changes in the histology of organs from PNA3 NPs treated mice were observed in comparison to the control group.

### Example 10: Enhancement of PNA Probes Using Gamma Modified PNAs (γPNAs)

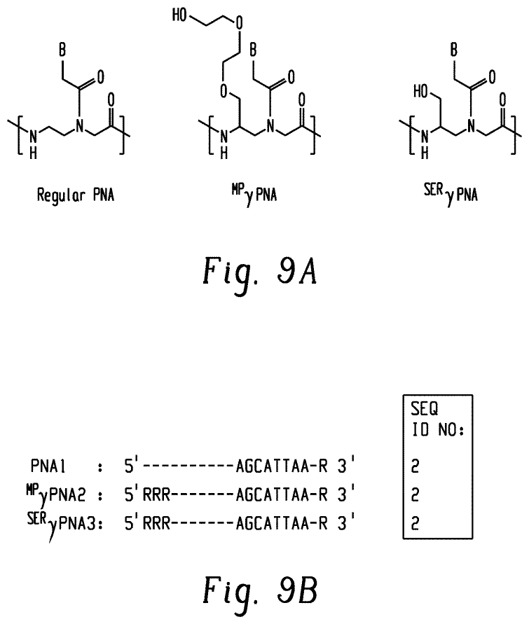

The therapeutic efficacy of proposed short PNA probes can be further enhanced by using next generation gamma modified PNAs (γPNAs). γPNAs are locked into right-handed helical structure due to stereogenic center at the gamma backbone position. γPNAs possess high binding affinity and improved water solubility as compared to regular PNAs. Hence, short cationic γPNAs were synthesized using miniPEG and serine modified gamma PNA monomers. FIG. 9 shows the chemical structures and sequences of regular PNA, MiniPEG-γPNA and serine-γPNA.

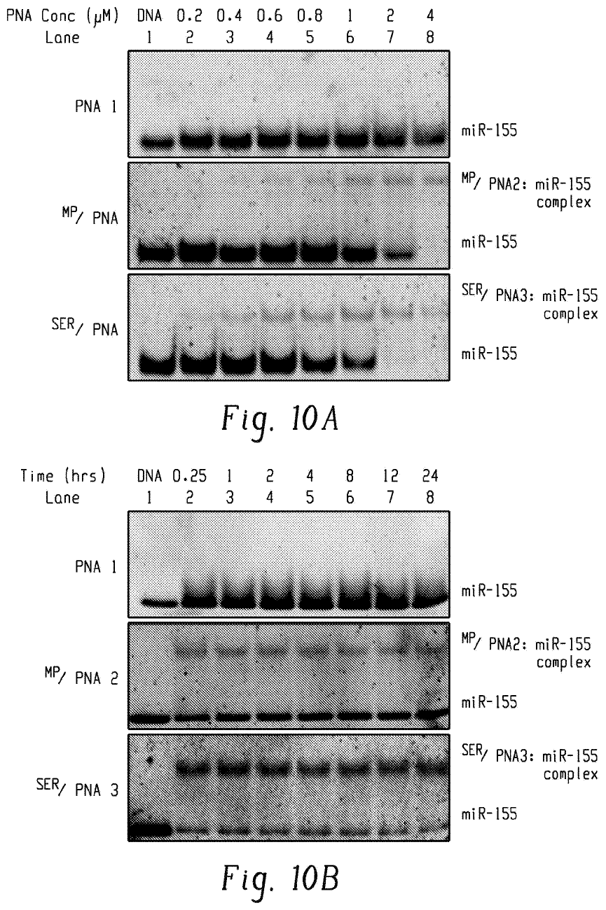

In order to investigate the binding affinity of gamma modified short cationic PNAs, binding studies were performed by incubating the PNAs with 23 mer miR-155 target at different concentrations in physiological conditions for 16 hours. Further the bound and unbound fractions were separated on a polyacrylamide gel and stained using SYBR gold. A retarded band was not observed at any concentrations after incubation of the target miR-155 with PNA1 (FIG. 10A, lane 8). However. ^{MP}γPNA2 showed a retarded band at 0.4 μM concentration (FIG. 10A, lane 3). Further, ^{MP}γPNA2 showed complete binding at 4 μM concentration as no unbound target miR-155 (Figure was seen in lane 8. Further. ^{Ser}γPNA3 showed binding to the target miR-155 at 0.2 μM concentration and complete binding was observed at 2 μM concentration. Hence, ^{Ser}γPNA3 shows superior binding affinity in comparison to ^{MP}γPNA2.

Further, the binding affinity of PNAs with the target miR-155 at different time points (FIG. 10B) to determine the binding kinetics. Two micromolar PNA were incubated with 1 μM of miR-155 at 37° C. for different time points from 0.25 hrs to 24 hrs. Higher intensity retarded bands were observed for ^{Ser}γPNA3 in comparison to ^{MP}vPNA2; however, PNA1 did not show any binding, consistent with the concentration dependent gel shift assay results. Hence, ^{Ser}γPNA3 exhibits superior binding affinity to the target miR-155 in comparison to ^{MP}γPNA2.

### Discussion of Results

Advances in the understanding of synthetic nucleic acid analogue chemistry in conjunction with optimization of their delivery strategies have certainly provided a new horizon for the translation of antisense oligonucleotides into the clinic. In particular, miRNA targeted drug candidates, Cobomarsen (targeting miR-155) and RG-012 (targeting miR-21) are in clinical trials for treatment of Cutaneous T cell Lymphoma and Alport syndrome respectively. Most of the present antimiR strategies are centered on targeting full length miRNAs (23mer) and subsequently inhibiting their interaction with the target mRNA. Herein, it was successfully demonstrated that targeting full length miRNAs by antimiRs is not essential for efficacy. A novel PNA design was tested; short PNAs containing three arginine amino acids on 5′ end (PNA3) that can target the seed sequence of miRNA-155 with enhanced binding affinity and inhibit its gene expression. Gel shift analysis demonstrated that both lysine as well as arginine containing short PNAs bind to miR-155 at low concentration as compared to non-cationic short PNA. Further, thermal denaturation results indicated that arginine containing short cationic PNAs possess superior binding affinity due to their Watson-Crick recognition between complementary sequences (seed region) and enhanced stabilization of the miRNA-PNA hetero-duplex via electrostatic interaction between negatively charged miRNA backbone (non-seed region) and the arginine containing positive domain. Though prior studies comprising poly-arginine conjugated PNAs (18mer) have shown promise to some extent, herein, only three arginine amino acids were chosen to minimize cytotoxicity induced by excess positive charge.

Though PNA has gained considerable attention as therapeutic agents, its intracellular delivery remains a challenge for their broader clinical applications. Prior strategies to increase PNAs traversing across the biological cell membrane include; a) conjugation to poly-arginine tails, and b) gamma modification of PNA backbone with guanidinium based transduction domains (also called guanidinium PNAs). However, these strategies possess a few unresolved challenges. The guanidinium PNA-based strategy demands high cost as it requires multistep synthesis for production of adequate amounts of guanidinium PNA monomers. Cytotoxicity associated with amphipathic polyarginine PNAs can limit its in vivo applications. In addition, for significant antisense effects, poly-arginine and guanidinium containing oligomers require longer incubation time and higher concentration of oligomers (due to endosomal entrapment). As an alternative, it has been demonstrated that the PLGA NPs provide an effective and safe strategy to deliver short cationic PNAs. Additionally, supported by comprehensive characterizations, PLGA NPs containing short cationic PNAs exhibit uniform morphology as well as size distribution. Further, it was observed sustained release of short cationic PNA3 in PLGA NPs.

The cell culture results illustrate that PNA8 loaded PLGA NPs undergo substantial uptake with uniform distribution in the cytosol of HeLa cells. Prior studies showed that PLGA NPs containing full length antimiR without cationic domain or polyarginine PNAs undergo significant localization in the endosomes. On the contrary, PNA8 containing PLGA NPs demonstrate greater transfection efficiency and undergo uniform distribution in the cytosol. Moreover, prior methods described that higher concentration of poly-arginine PNAs or guanidinium PNAs are required for cellular uptake. Here, PNA8 alone at small concentrations do not show significant transfection in HeLa cells. Arginine containing short PNA8 NPs show uniform cellular distribution were also examined as compared to non-arginine containing PNA6 NPs or PNA10 NPs. Two plausible explanations for the aforementioned observations are: a) short cationic PNA NPs could undergo substantial solvent penetration that results in faster polymer degradation as compared to full length antimiR PNA containing NPs, resulting in uniform intracellular distribution, and b) arginine residues in short PNAs also assist in endosomal escape due to their interaction with negatively charged endosomal membranes.

Further, these gene expression results indicate that arginine containing short cationic PNAs result in increased miR-155 knockdown as compared to lysine containing short PNAs. Without being bound by theory, one possible explanation of higher binding affinity of the arginine versus lysine containing group is that arginine is more positively charged as compared to lysine. Hence arginine containing PNAs can undergo extensive electrostatic interaction with the phosphate clusters of the RNA backbone leading to enhanced binding and subsequently superior antimiR efficacy.

Herein, it has been confirmed that short PNAs containing arginine domains can efficiently target the seed region of miR-155 and inhibit its expression. Further, in conjunction with nanotechnology, it was determined that PLGA NPs can be used to encapsulate short cationic PNAs with superior physico-biochemical properties for gene therapy applications. The design described herein offers potential benefits in terms of low cost, better efficacy, minimal cytotoxicity, and is clinically translatable. Moreover, non-selective binding and off-target effects of short PNAs can be minimized due to promising delivery strategies since PLGA NPs target the tumor by enhanced permeability and retention (EPR) effect. Inclusive transcriptional and proteomic profiling based on tiny LNA based studies suggested that tiny LNAs targeting the seed region have minimal off-target effects and does not significantly affect the other mRNAs gene expression even without using delivery systems. It is believe that short cationic PNAs with an optimized PLGA based delivery system will further improve the anti-seed based anti-miR technology with minimal genomic off-target toxicity and adverse reactions.

Finally, supported by comprehensive end point analysis, evidence has been provided that short anti-seed cationic PNAs delivered by PLGA NPs are safe and non-toxic after systemic administration in the immunocompetent mice. These indications of safety are in line with previous work that has established PNAs and PLGA NPs with favorable side effect profiles. It also has been established that PLGA NPs containing short PNAs inhibit miR-155 expression after systemic delivery. Importantly, the PLGA NPs containing PNA3 showed tumor growth inhibition as compared to only PNA3 and controls. Further, PNA3 NPs showed similar or superior efficacy in reducing the tumor growth in comparison to full length PNA5 NPs. In prior studies, full length antimiR-155 PNAs in a lymphoma mouse model showed considerable anti-tumor activity. In one case, antimiR-155 PNAs were encapsulated and cell penetrating peptide coated PLGA NPs were used to increase tumor accumulation and targeting. Whereas in another case, PNAs were delivered to tumor microenvironment by conjugation with tumor selective pH sensitive peptide known as pH low insertion peptide (pHLIP).

The results provided herein establish that short cationic PNAs can effectively inhibit an oncogenic miRNA by simple intravenous infusion of regular PLGA NPs without any ligand coating or chemical modification. Though we noticed optimal effects in tumor growth inhibition, the efficacy of our system could be improved through a number of methods. First, next generation gamma modified PNAs (γPNAs) can be used to further increase the therapeutic efficacy of short PNAs. γPNAs are locked into right-handed helical structure due to stereogenic center at the gamma backbone position. γPNAs possess high binding affinity and improved water solubility as compared to regular PNAs. Prior studies demonstrated that γPNAs can be used as effective antisense and gene editing agents. In addition, the enhanced activity of γPNAs can be further augmented by next generation pHLIP and ligand coated delivery systems.

Together, these results establish that short anti-seed PNAs can be used as effective therapeutic modality for targeting miRNAs.

The use of the terms “a,” “an,” “the,” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising.” “having,” “including,” and “containing” are to be construed as open-ended terms (i.e., meaning “including, but not limited to”) unless otherwise noted. “About” or “approximately,” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±10% or 5% of the stated value. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A modified anti-seed PNA comprising 5′-Xaa1Xaa2Xaa3-N1N2N3N4N5N6N7N8 N9-Xaa4-3′, wherein Xaa1, Xaa2, Xaa3, and Xaa4 are each independently R or K, and wherein N1N2N3N4N5N6N7N8 N9 is a PNA that Watson-Crick base pairs to a seed sequence of a miRNA, wherein N8 and N9 may be null.

2. The modified anti-seed PNA of claim 1, wherein Xaa1, Xaa2, Xaa3, and Xaa4 are R, or wherein Xaa1, Xaa2, Xaa3, and Xaa4 are K.

3. The modified anti-seed PNA of claim 1, wherein the miRNA is let-7b, let-7c, let-7d, let-7e, let-7f, let-7g, let-7i, miR-100, miR-103, miR-106a, miR-107, miR-10a, miR-10b, miR-122, miR-125a, miR-125b, miR-126, miR-126*, miR-127-3p, miR-128a, miR-129, miR-133b, miR-135b, miR-137, miR-141, miR-143, miR-145, miR-146a, miR-146b, miR-148a, miR-149, miR-150, miR-155, miR-15a, miR-17-3p, miR-17-5p, miR-181a, miR-181b, miR-181c, miR-183, miR-184, miR-186, miR-187, miR-189, miR-18a, miR-190, miR-191, miR-192, miR-197, miR-199a, miR-199a*, miR-19a, miR-19b, miR-200a, miR-200a*, miR-200b, miR-200c, miR-202, miR-203, miR-205, miR-20a, miR-21, miR-210, miR-216, miR-218, miR-22, miR-221, miR-222, miR-223, miR-224, miR-23a, miR-23b, miR-24, miR-25, miR-26a, miR-26b, miR-27a, miR-27b, miR-29a, miR-29b, miR-296-5p, miR-301, miR-302a, miR-302a*, miR-30a, miR-30b, miR-30c, miR-30d, miR-30e-3p, miR-30e-5p, miR-31, miR-320, miR-323, miR-324-5p, miR-326, miR-330, miR-331, miR-335, miR-346, miR-34a, miR-370, miR-372, miR-373, miR-373*, miR-497, miR-498, miR-503, miR-92, miR-93, miR-96, or miR-99a.

4. The modified anti-seed PNA of claim 1, wherein the miRNA is miR-155

5. The modified anti-seed PNA of claim 1, wherein N9 is null.

6. The modified anti-seed PNA of claim 5, wherein N1N2N3N4N5N6N7N8 is AGCATTAA.

7. The modified anti-seed PNA of claim 1, further comprising a detectable label, such as a 5′ TAMRA with an 8-amino-2,6,10-trioxaoctanoic acid linker.

8. The modified anti-seed RNA of claim 1, wherein the PNA is a gamma modified PNA.

9. The modified anti-seed PNA of claim 1, encapsulated in a nanoparticle.

10. The modified anti-seed PNA of claim 9, wherein the nanoparticle comprises polylactic-co-glycolic acid (PLGA), polylactic acid (PLA), polyglycolic acid (PGA), chitosan, gelatin, polycaprolactone, a poly-alkyl-cyanoacrylate, mesoporous silica nanoparticles, Poly-beta-amino-esters, polyethyleneimine (PEI), or a combination thereof.

11. A pharmaceutical composition comprising the anti-seed PNA of claim 1 and a pharmaceutically acceptable excipient.

12. A method of inhibiting expression of a miRNA in vivo or in vitro, comprising contacting a cell with an inhibitory amount of the modified anti-seed PNA of claim 1.

13. The method of claim 12, wherein the cell is a tumor cell.

14. A method of treating cancer, comprising administering to a subject in need thereof the anti-seed PNA of claim 1.

15. The method of claim 14, wherein the cancer is lymphoma, leukemia, breast, colon or lung cancer.

16. A method of treating cardiovascular disease, inflammation, stroke, Alzheimer's disease, schizophrenia, a lysosomal storage disorder or progeria, comprising administering to a subject in need thereof the anti-seed PNA of claim 1.


SEQUENCE LISTING (33 sequences)

SEQ ID NO: 1 - DNA, 12 bp, Artificial Sequence
  Based on Homo sapiens sequence
kkkagcattaak

SEQ ID NO: 2 - DNA, 12 bp, Artificial Sequence
  Based on Homo sapiens sequence
rrragcattaar

SEQ ID NO: 3 - DNA, 12 bp, Artificial Sequence
  Based on Homo sapiens sequence
rrrtaacgatar

SEQ ID NO: 4 - DNA, 27 bp, Artificial Sequence
  Based on Homo sapiens sequence
rrracccctatcacgattagcattaar

SEQ ID NO: 5 - RNA, 22 nt, Homo sapiens
uagcuuaucagacugauguuga

SEQ ID NO: 6 - RNA, 24 nt, Homo sapiens
uuaaugcuaaucgugauagggguu

SEQ ID NO: 7 - RNA, 22 nt, Homo sapiens
accuggcauacaauguagauuu

SEQ ID NO: 8 - RNA, 21 nt, Homo sapiens
uacucaaaaagcugucaguca

SEQ ID NO: 9 - RNA, 23 nt, Homo sapiens
uacccuguagaaccgaauuugug

SEQ ID NO: 10 - RNA, 22 nt, Homo sapiens
uggaaacauuucugcacaaacu

SEQ ID NO: 11 - RNA, 22 nt, Homo sapiens
uggagugugacaaugguguuug

SEQ ID NO: 12 - RNA, 22 nt, Homo sapiens
aguucuucaguggcaagcuuua

SEQ ID NO: 13 - RNA, 22 nt, Homo sapiens
aacccguagauccgaucuugug

SEQ ID NO: 14 - RNA, 23 nt, Homo sapiens
cggggccguagcacugucugaga

SEQ ID NO: 15 - RNA, 24 nt, Homo sapiens
uuuggcaaugguagaacucacacu

SEQ ID NO: 16 - RNA, 22 nt, Homo sapiens
accuggcauacaauguagauuu

SEQ ID NO: 17 - RNA, 22 nt, Homo sapiens
cucaguagccaguguagauccu

SEQ ID NO: 18 - RNA, 16 nt, Homo sapiens
cuaggaggccuuggcc

SEQ ID NO: 19 - RNA, 23 nt, Homo sapiens
uaaagugcuuauagugcagguag

SEQ ID NO: 20 - RNA, 22 nt, Homo sapiens
ucccugagacccuaacuuguga

SEQ ID NO: 21 - RNA, 23 nt, Homo sapiens
caaagugcuuacagugcagguag

SEQ ID NO: 22 - RNA, 21 nt, Homo sapiens
cauaaaguagaaagcacuacu

SEQ ID NO: 23 - RNA, 23 nt, Homo sapiens
aacauucaacgcugucggugagu

SEQ ID NO: 24 - RNA, 22 nt, Homo sapiens
ggcuacaacacaggacccgggc

SEQ ID NO: 25 - RNA, 22 nt, Homo sapiens
uguaaacauccucgacuggaag

SEQ ID NO: 26 - DNA, 23 bp, Artificial Sequence
  Based on Homo sapiens sequence
ttaatgctaatcgtgataggggt

SEQ ID NO: 27 - DNA, 15 bp, Artificial Sequence
  Based on Homo sapiens sequence
  misc_feature (1)..(1): 5-Carboxytetramethylrhodamine (TAMRA)
  misc_feature (2)..(4): 8-amino-2,6,10-trioxaoctanoic acid residues (Mini-PEG)
nnnnrrragcattaa

SEQ ID NO: 28 - DNA, 16 bp, Artificial Sequence
  Based on Homo sapiens sequence
  misc_feature (1)..(1): 5-Carboxytetramethylrhodamine (TAMRA)
  misc_feature (2)..(4): 8-amino-2,6,10-trioxaoctanoic acid residues (Mini-PEG)
nnnnkkkagcattaak

SEQ ID NO: 29 - DNA, 16 bp, Artificial Sequence
  Based on Homo sapiens sequence
  misc_feature (1)..(1): 5-Carboxytetramethylrhodamine (TAMRA)
  misc_feature (2)..(4): 8-amino-2,6,10-trioxaoctanoic acid residues (Mini-PEG)
nnnnrrragcattaar

SEQ ID NO: 30 - DNA, 16 bp, Artificial Sequence
  Based on Homo sapiens sequence
  misc_feature (1)..(1): 5-Carboxytetramethylrhodamine (TAMRA)
  misc_feature (2)..(4): 8-amino-2,6,10-trioxaoctanoic acid residues (Mini-PEG)
nnnnrrrtaacgatar

SEQ ID NO: 31 - DNA, 28 bp, Artificial Sequence
  Based on Homo sapiens sequence
  misc_feature (1)..(1): 5-Carboxytetramethylrhodamine (TAMRA)
  misc_feature (2)..(4): 8-amino-2,6,10-trioxaoctanoic acid residues (Mini-PEG)
nnnnacccctatcacgattagcattaar

SEQ ID NO: 32 - DNA, 31 bp, Artificial Sequence
  Based on Homo sapiens sequence
  misc_feature (1)..(1): 5-Carboxytetramethylrhodamine (TAMRA)
  misc_feature (2)..(4): 8-amino-2,6,10-trioxaoctanoic acid residues (Mini-PEG)
nnnnrrracccctatcacgattagcattaar

SEQ ID NO: 33 - DNA, 12 bp, Artificial Sequence
  Based on Homo sapiens sequence
rrragcattaar